(19) United States
(12) Patent Application Publication
(10) Pub. No.: US 2025/0241910 A1
(43) Pub. Date: Jul. 31, 2025

(54) COMBINATION THERAPY FOR THE TREATMENT OF PSYCHIATRIC DISORDER

(71) Applicant: GSKB Pharmaceuticals, LLC, Naperville, IL (US)

(72) Inventors: Sandeep Gaonkar, Naperville, IL (US); Kara Mulligan, Naperville, IL (US); Paul Sudhakar, Shawnee, KS (US); Scott Boyer, West Chicago, IL (US)

(21) Appl. No.: 19/067,074

(22) Filed: Feb. 28, 2025

Related U.S. Application Data
(63) Continuation of application No. 18/633,291, filed on Apr. 11, 2024.
(60) Provisional application No. 63/495,698, filed on Apr. 12, 2023.

(51) Int. Cl. A61K 31/496; A61K 9/20; A61K 31/519

(52) U.S. Cl. CPC A61K 31/496; A61K 9/2009; A61K 9/2013; A61K 9/2018; A61K 9/2054; A61K 31/519

(57) ABSTRACT

The present invention provides for a method of treating a person suffering from a psychiatric disorder that includes administration of a pharmaceutically effective amount of (i) nefazodone, or a pharmaceutically acceptable salt thereof, and (ii) risperidone. Also provided is a unit dose that includes (i) nefazodone, or a pharmaceutically acceptable salt thereof, (ii) risperidone, and (iii) one or more pharmaceutically acceptable excipients. Also provided is a therapeutic package that includes (a) the unit doses, (b) a finished pharmaceutical container that contains the unit doses, (c) printed indicia, and (d) optionally an outside container containing the finished pharmaceutical container and the printed indicia.

### CROSS REFERENCE TO RELATED APPLICATIONS

The present application is a continuation of U.S. patent application Ser. No. 18/633,291, filed on Apr. 11, 2024, which claims the benefit of priority to U.S. Provisional Application No. 63/495,698, filed Apr. 12, 2023, the contents of which are hereby incorporated by reference.

### BACKGROUND OF THE INVENTION

Suicide is death caused by injuring oneself with the intent to die. A suicide attempt is when someone harms themselves with any intent to end their life, but they do not die as a result of their actions.¹

Many factors can increase the risk for suicide or protect against it. Suicide is connected to other forms of injury and violence. For example, people who have experienced violence, including child abuse, bullying, or sexual violence have a higher suicide risk. Being connected to family and community support and having easy access to healthcare can decrease suicidal thoughts and behaviors.²

Suicide is a serious public health problem. Suicide rates increased approximately 36% between 2000-2021. Suicide was responsible for 48,183 deaths in 2021, which is about one death every 11 minutes.³ The number of people who think about or attempt suicide is even higher. In 2021, an estimated 12.3 million American adults seriously thought about suicide, 3.5 million planned a suicide attempt, and 1.7 million attempted suicide.⁴

Suicide affects people of all ages. In 2021, suicide was among the top 9 leading causes of death for people ages 10-64. Suicide was the second leading cause of death for people ages 10-14 and 20-34.³

Some groups have higher suicide rates than others. Suicide rates vary by race/ethnicity, age, and other factors, such as where someone lives. By race/ethnicity, the groups with the highest rates are non-Hispanic American Indian/Alaska Native people followed by non-Hispanic White people.³ Other Americans with higher-than-average rates of suicide are veterans, people who live in rural areas, and workers in certain industries and occupations like mining and construction.^{5,6} Young people who identify as lesbian, gay, or bisexual have higher prevalence of suicidal thoughts and behavior compared to their peers who identify as heterosexual.⁷

Suicide has far-reaching impact. Suicide and suicide attempts cause serious emotional, physical, and economic impacts. People who attempt suicide and survive may experience serious injuries that can have long-term effects on their health. They may also experience depression and other mental health concerns.⁸

Suicide and suicide attempts affect the health and well-being of friends, loved ones, co-workers, and the community. When people die by suicide, their surviving family and friends may experience prolonged grief, shock, anger, guilt, symptoms of depression or anxiety, and even thoughts of suicide themselves.^{8.9}

The financial toll of suicide on society is also costly. In 2020, suicide and nonfatal self-harm cost the nation over $500 billion in medical costs, work loss costs, value of statistical life, and quality of life costs.

### REFERENCES

1. Crosby A, Ortega L, Melanson C. Self-directed violence surveillance: Uniform definitions and recommended data elements, version 1.0. (2011) Atlanta, GA: Centers for Disease Control and Prevention, National Center for Injury Prevention and Control.
2. CDC. Preventing multiple forms of violence: A strategic vision for connecting the dots. (2016) Atlanta, GA: Centers for Disease Control and Prevention, National Center for Injury Prevention and Control.
3. Centers for Disease Control and Prevention, National Center for Health Statistics. National Vital Statistics System, Mortality 2018-2021 on CDC WONDER Online Database, released in 2023. Data are from the Multiple Cause of Death Files, 2018-2021, as compiled from data provided by the 57 vital statistics jurisdictions through the Vital Statistics Cooperative Program. Accessed at http://wonder.cdc.gov/mcd-icd10-expanded.html on Jan. 11, 2023.
4. Substance Abuse and Mental Health Services Administration (2022). Key substance use and mental health indicators in the United States: Results from the 2021 National Survey on Drug Use and Health (HHS Publication No. PEP22-07-01-005, NSDUH Series H-57). Center for Behavioral Health Statistics and Quality, Substance Abuse and Mental Health Services Administration. https://www.samhsa.gov/data/report/2021-nsduh-annual-national-report.
5. Centers for Disease Control and Prevention. (2022). Suicide Prevention Resource for Action: A Compilation of the Best Available Evidence. Atlanta, GA: National Center for Injury Prevention and Control, Centers for Disease Control and Prevention.
6. Peterson C, Sussell A, Li J, Schumacher P, Yeoman K, Stone D. (2020) Suicide Rates by Industry and Occupation—National Violent Death Reporting System, 32 States, 2016. MMWR Morb Mortal Wkly Rep; 69:57-62. DOI: http://dx.doi.org/10.15585/mmwr.mm6903al.
7. Centers for Disease Control and Prevention (CDC). Youth Risk Behavior Survey Data Summary and Trends Report: 2011-2021. Available at https://www.cdc.gov/healthyyouth/data/yrbs/pdf/YRBS_Data-Summary-Trends_Report2023_508.pdf.
8. Hamdan, S., Berkman, N., Lavi, N., Levy, S., Brent, D. (2020). The effect of sudden death bereavement on the risk for suicide. Crisis; 41 (3): 214-224. DOI: https://doi.org/10.1027/0227-5910/a000635.
9. Wagner, B., Hofmann, L., Grafiadeli, R. (2021). The relationship between guilt, depression, prolonged grief, and posttraumatic stress symptoms after suicide bereavement. J Clin Psychol; 77:2545-2558. DOI: https://doi.org/10.1002/jclp.23192.

### SUMMARY OF THE INVENTION

In the treatment of patients with numerous drug therapies, the combination product described herein (as two separate dosage forms) shows significant benefit in the reduction of suicide ideation. Without being bound to any particular theory, the present inventors believe that the nefazodone component of the medication reduces suicide ideation in patients and the risperidone component reduces the impulsivity component or “willingness to act” in patients with suicide ideation. The combination of the two active ingredients allows the patient to live a much more normal life by reducing their suicide ideation and their willingness to act on their suicide ideation.

The present invention relates to the use of (i) nefazodone, or a pharmaceutically acceptable salt thereof, and (ii) risperidone, for the treatment of persons suffering from a psychiatric disorder (e.g., suicidal behavior, suicidal ideation, and/or major depressive disorder (MDD)). This includes persons at risk of committing suicide, persons having recently attempted to commit suicide, and/or persons having a history of suicide attempts. This also includes persons that are afflicted with treatment-refractory depression.

The present invention provides for a method that includes administering to a person a unit dose that includes nefazodone hydrochloride, risperidone, and one or more pharmaceutically acceptable excipients.

The present invention also provides for a method for the treatment of a psychiatric disorder. The method includes administering to the person a unit dose that includes nefazodone or a pharmaceutically acceptable salt thereof, risperidone, and one or more pharmaceutically acceptable excipients.

The present invention also provides for a method of treating a person suffering from a psychiatric disorder. The method includes: (a) marketing to prescribers a pharmaceutical product containing (i) nefazodone, or a pharmaceutically acceptable salt thereof and (ii) risperidone, as being effective for the treatment of person suffering from a psychiatric disorder; (b) prescribing the pharmaceutical product to a patient having been diagnosed by the physician, as suffering from the psychiatric disorder, in response to the marketing of the pharmaceutical product and the diagnosis of the patient; and (c) administering the prescribed pharmaceutical product to the patient.

The present invention also provides for a method of treating a person suffering from a psychiatric disorder. The method includes (a) providing to the marketplace a pharmaceutical product containing (i) nefazodone, or a pharmaceutically acceptable salt thereof and (ii) risperidone; (b) sensitizing a physician to a product claim that the pharmaceutical product is effective for the treatment of person suffering from the psychiatric disorder; (c) selectively prescribing the pharmaceutical product to the patient having been diagnosed by the sensitized physician, as suffering from the psychiatric disorder, in view of the diagnosis of the patient; and (d) orally administering the prescribed pharmaceutical product to the patient.

The present invention also provides for a method for treating a patient suffering from a psychiatric disorder. The method includes (a) purchasing a pharmaceutical product containing (i) nefazodone, or a pharmaceutically acceptable salt thereof and (ii) risperidone; (b) tendering a prescription for the patient suffering from the psychiatric disorder before completing the purchase transaction, the prescription having been received in response to a diagnosis of the psychiatric disorder by a physician, the physician (1) having been sensitized to a product claim that the pharmaceutical product is effective for the treatment of person suffering from the psychiatric disorder, and (2) having selectively prescribed the pharmaceutical product; and (c) administering the purchased pharmaceutical product to the patient.

The present invention also provides for a unit dose that includes: (i) nefazodone, or a pharmaceutically acceptable salt thereof, (ii) risperidone, and (iii) one or more pharmaceutically acceptable excipients.

The present invention also provides for a therapeutic package that includes: (a) unit doses that include: (i) nefazodone, or a pharmaceutically acceptable salt thereof, (ii) risperidone, and (iii) one or more pharmaceutically acceptable excipients, (b) a finished pharmaceutical container that contains the unit doses, (c) written matter or printed indicia associated with the therapeutic package stating that the unit doses can be administered to treat a psychiatric disorder in a person, and (d) optionally an outside container containing the finished pharmaceutical container.

The present invention also provides for a therapeutic package for dispensing unit doses to a person being treated for a psychiatric disorder. The therapeutic package includes: (a) one or more unit doses, each unit dose including an effective amount of (i) nefazodone, or a pharmaceutically acceptable salt thereof, (ii) risperidone, and (iii) one or more pharmaceutically acceptable excipients, to treat the psychiatric disorder; and (b) a container containing: (i) the unit dose or unit doses and (ii) labeling directing the use of the package, unit dose, or unit doses in the treatment of the psychiatric disorder in a person.

### DETAILED DESCRIPTION OF THE INVENTION

The present invention relates to the psychotherapeutic combination of (i) nefazodone, or a pharmaceutically acceptable salt thereof, and (ii) risperidone. The psychotherapeutic combination is present within the same unit dose, along with (iii) one or more pharmaceutically acceptable excipients. The unit doses can be contained within a finished pharmaceutical container. Together with written matter or printed indicia, the finished pharmaceutical container can optionally be contained within an outside container. The unit dose can be administered to a person for the treatment of a psychiatric disorder. The psychiatric disorder can include, e.g., suicidal behavior, suicidal ideation, and/or major depressive disorder (MDD). This includes persons at risk of committing suicide, persons having recently attempted to commit suicide, and/or persons having a history of suicide attempts.

The use of the terms “a” and “an” and “the” and similar referents in the context of describing the invention are to be construed to cover both the singular and the plural, unless otherwise indicated herein or clearly contradicted by context.

The description herein of any aspect or aspect of the invention using terms such as “comprising”, “having,” “including” or “containing” with reference to an element or elements is intended to provide support for a similar aspect or aspect of the invention that “consists of”, “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the various aspects, embodiments, implementations, and features of the invention mentioned herein may be claimed separately, or in any combination.

### Definitions

Throughout the description, the term “nefazodone” refers to refers to the compound 1-(3-[4-(3-chlorophenyl)piperazin-1-yl]propyl)-3-ethyl-4-(2-phenoxyethyl)-1H- 1,2,4-triazol-5(4H)-one. The chemical structure is shown below.

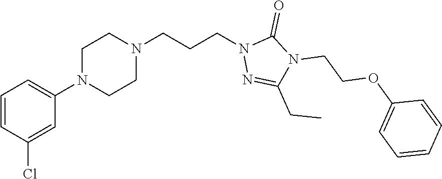 [SMILES: CCc1nn(CCCN2CCN(c3cccc(Cl)c3)CC2)c(=O)n1CCOc1ccccc1]

Nefazodone can also exist in the salt form (e.g., nefazodone hydrochloride). Nefazodone is an antidepressant and has the CAS number 83366-66-9 (82752-99-6 as the hydrochloride), unique ingredient identifier (UNII) 59H4FCVITF, chemical formula C₂₅H₃₂ClN₅O₂, and molar mass 470.01 g·mol⁻¹. Nefazodone is a selective serotonin 5-HT₂ receptor antagonist. Preparation: D. L. Temple, Jr., W. G. Lobeck, Jr., U.S. Pat. No. 4,338,317 (1982 to Mead Johnson). Synthesis and x-ray crystal structure: G. D. Madding et al, J. Heterocycl. Chem. 22, 1121 (1985). Pharmacology: A. S. Eison et al., Psychopharmacol. Bull. 26, 311 (1990). HPLC determination in plasma: J. E. Franc et al., J. Chromatogr. 570, 129 (1991). Clinical trial in depression: M. F. D'Amico et al., Psychopharmacol. Bull. 26, 147 (1990); in combination with psychotherapy for chronic depression: M. B. Keller et al., N. Engl. J. Med. 342, 1462 (2000). Review: W. E. Heydorn, Expert Opin. Invest. Drugs 4, 131-137 (1995). The FDA (3Q 2022) provides for the following drug master file (DMF) holders: Omnichem SA (Nefazodone II), Esteve Quimica SA (Nefazodone Hydrchloride), TEVA Pharmaceutical Industries LTD (Nefazodone Hydrochloride USP), and Signa SA DE CV (Nefazodone Hydrochloride). Throughout the description, the term “nefazodone,” without specification of any particular salt form, is intended to include any form of the compound, such as the free base and pharmaceutically acceptable salts. The free base and pharmaceutically acceptable salts include anhydrous forms and solvated forms such as hydrates. The anhydrous forms and the solvates include amorphous and crystalline forms. In a particular embodiment, nefazodone is in the form of the hydrochloride salt and is denoted “nefazodone hydrochloride” or “nefazodone HCl”.

Throughout the description, the term “risperidone” refers to the compound 3-[2-[4-(6-fluoro-1,2-benzoxazol-3-yl) piperidin-1-yl] ethyl]-2-methyl-6,7,8,9- tetrahydropyrido [1,2-a]pyrimidin-4-one. The chemical structure is shown below.

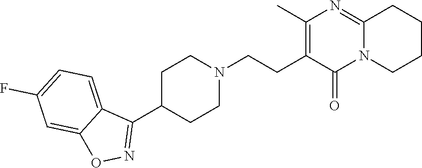 [SMILES: Cc1nc2n(c(=O)c1CCN1CCC(c3noc4cc(F)ccc34)CC1)CCCC2]

Risperidone is an antipsychotic and has the CAS number 106266 Jun. 2, unique ingredient identifier (UNII) L6UH7ZF8HC, chemical formula C₂₃H₂₇FN₄O₂, and molar mass 410.493 g·mol⁻¹. Risperidone is a combined serotonin (5-HT2) and dopamine (D₂) receptor antagonist. Preparation: L. E. J. Kennis, J. Vandenberk, EP 196132; eidem, U.S. Pat. No. 4,804,663 (1986, 1989 both to Janssen). Pharmacology: P. A. J. Janssen et al., T. Pharmacol. Exp. Ther. 244, 685 (1988). Receptor binding studies: J. E. Leysen et al., ibid. 247, 661 (1988). HPLC determination in plasma: A. Avenoso et al., J. Chromatogr. B 746, 173 (2000). Clinical study in psychoses: Y. G. Gelders et al., Pharmacopsychiatry 23, 206 (1990); in autism: L. Scahill et al., N. Engl. J. Med. 347, 314 (2002). Brief review: M. G. Livingston, Lancet 343, 457-460 (1994). Review of pharmacology and therapeutic potential: S. Grant, A. Fitton, Drugs 48, 253-273 (1994); B. Green, Curr. Med. Res. Opin. 16, 57-65 (2000); of clinical experience in schizophrenia: H.-J. Moller, Expert Opin. Pharmacother. 6, 803-818 (2005). The FDA (3Q 2022) provides for the following drug master file (DMF) holders: Janssen Pharmaceutica NV (Risperidone (R064766) Drug Substance), TEVA Pharmaceutical Industries LTD (Risperidone USP), INKE SA (Risperidone), Dr. Reddy's Laboratories LTD (Risperidone EP), and Medichem SA (Risperidone). Throughout the description, the term “risperidone,” without specification of any particular physical form, is intended to include any form of the compound, such as anhydrous forms and solvated forms such as hydrates. The anhydrous forms and the solvates include amorphous and crystalline forms.

In the present context, the terms “psychiatric disorder” and “mental disorder” encompass disorders, conditions, and accompanying symptoms associated with those listed in DSM-5-TR (Diagnostic and Statistical Manual of Mental Disorders—5th Ed., Am. Psych. Association). These include, e.g., schizophrenia and other psychotic disorders, depressive disorders (e.g., major depressive disorder), bipolar disorders, as well as accompanying suicidal behavior and/or suicidal ideation. The psychiatric disorder can last a finite period of time. Alternatively, the psychiatric disorder can be chronic, such that it is persistent or otherwise long-lasting. When chronic, the psychiatric disorder can even be lifelong, with recurring episodes.

In the present context, the term “suicidal behavior” refers to performing actions that put oneself at risk of death. Suicidal behavior may include acts of self-harm with a fatal (completed suicide) or a nonfatal (attempted suicide) outcome. The Columbia-Suicide Severity Rating Scale (C-SSRS) (http://www.cssrs.columbia.edu), one of several available suicidal behavior instruments, defines five subtypes or levels of suicidal behavior considered important to capture in any prospective assessment. These include (i) completed suicide, (ii) suicide attempt, (iii) interrupted attempt, (iv) aborted attempt, and (v) preparatory actions toward imminent suicidal behaviors.

In the present context, the term “completed suicide” refers to a type of suicidal behavior and is a self-injurious behavior that resulted in fatality and was associated with at least some intent to die as a result of the act. Evidence that the individual intended to kill him-or herself, at least to some degree, can be explicit or inferred from the behavior or circumstance.

In the present context, the term “suicide attempt” refers to a type of suicidal behavior and is a potentially self-injurious behavior, associated with at least some intent to die as a result of the act. Evidence that the individual intended to kill him-or herself, at least to some degree, can be explicit or inferred from the behavior or circumstance. A suicide attempt may or may not result in actual injury.

In the present context, the term “interrupted suicide attempt” refers to a type of suicidal behavior and occurs when the person is interrupted (by an outside circumstance) from starting a potentially self-injurious act (if not for that, actual attempt would have occurred).

In the present context, the term “aborted suicide attempt” refers to a type of suicidal behavior and occurs when the person begins to take steps toward making a suicide attempt, but stops before actually engaging in any self-destructive behavior. Examples are similar to interrupted attempts, except that the individual stops before being stopped by something else.

In the present context, the term “preparatory acts toward imminent suicidal behaviors” refers to self-injurious behavior associated with no intent to die. The behavior is intended purely for other reasons, either to relieve distress (often referred to as self-mutilation (e.g., superficial cuts or scratches, hitting or banging, or burns)) or to effect change in others or the environment.

In the present context, the term “suicidal ideation” or “suicidal thoughts” refers a person having thoughts, ideas, or ruminations about the possibility of ending one's own life. The DSM-5 defines it as “thoughts about self-harm, with deliberate consideration or planning of possible techniques of causing one's own death”. The Centers for Disease Control and Prevention defines suicidal ideation “as thinking about, considering, or planning suicide”. The ICD-11 describes suicidal ideation as “thoughts, ideas, or ruminations about the possibility of ending one's life, ranging from thinking that one would be better off dead to formulation of elaborate plans.” It is not a diagnosis but is a symptom of some mental disorders and can also occur in response to adverse events without the presence of a mental disorder. On suicide risk scales, the range of suicidal ideation varies from fleeting thoughts to detailed planning. Passive suicidal ideation is thinking about not wanting to live or imagining being dead. Active suicidal ideation is thinking about different ways to die or forming a plan to die. Suicidal ideation is associated with depression and other mood disorders; however, many other mental disorders, life events, and family events, can increase the risk of suicidal ideation. Mental health researchers indicate that healthcare systems should provide treatment for individuals with suicidal ideation, regardless of diagnosis, because of the risk for suicidal acts and repeated problems associated with suicidal thoughts. Suicidal ideation is a symptom for many mental disorders and can occur in response to adverse life events without the presence of a mental disorder. There are several psychiatric disorders that appear to be comorbid with suicidal ideation or considerably increase the risk of suicidal ideation. For example, many individuals with borderline personality disorder exhibit recurrent suicidal behavior and suicidal thoughts. The following list includes the disorders that have been shown to be the strongest predictors of suicidal ideation. These are not the only disorders that can increase risk of suicidal ideation. The disorders in which risk is increased the greatest include: anxiety disorders, autism spectrum disorder, major depressive disorder, dysthymia, bipolar disorder, attention deficit hyperactivity disorder (ADHD), premenstrual dysphoric disorder (PMDD), post-traumatic stress disorder (PTSD) and complex post-traumatic stress disorder (C-PTSD), personality disorders, psychosis (detachment from reality, paranoia, catatonia, hallucinations, etc.), schizophrenia, substance use disorders, body dysmorphic disorder, nightmare disorder, gender dysphoria, conduct disorder, and specific learning disorder. The Columbia-Suicide Severity Rating Scale (C-SSRS) (http://www.cssrs.columbia.edu), one of several available suicidal ideation instruments, defines five subtypes or levels of suicidal ideation considered important to capture in any prospective assessment. These include (i) passive, (ii) active: nonspecific (no method, intent, or plan), (iii) active: method, but no intent or plan, (iv) active: method and intent, but no plan, and (v) active: method, intent, and plan.

In the present context, the term “passive suicidal ideation: wish to be dead” refers to a type of suicidal ideation in which the person has thoughts about a wish to be dead or not alive anymore, or wish to fall asleep and not wake up.

In the present context, the term “active suicidal ideation: nonspecific (no method, intent, or plan)” refers to a type of suicidal ideation in which the person has general nonspecific thoughts of wanting to end one's life or commit suicide (e.g., “I've thought about killing myself”) without general thoughts of ways to kill oneself/associated methods, intent, or plan during the assessment period.

In the present context, the term “active suicidal ideation: method, but no intent or plan” refers to a type of suicidal ideation in which the person has thoughts of suicide and has thought of at least one method during the assessment period. This situation is different than a specific plan with time, place, or method details worked out (e.g., thought of method to kill self but not a specific plan). It includes when person who would say, “I thought about taking an overdose but I never made a specific plan as to when, where, or how I would actually do it . . . and I would never go through with it.”

In the present context, the term “active suicidal ideation: method and intent, but no plan” refers to a type of suicidal ideation in which the person has active suicidal thoughts of killing oneself, and the person reports having some intent to act on such thoughts, as opposed to “I have the thoughts but I definitely will not do anything about them.”

In the present context, the term “active suicidal ideation: method, intent, and plan” refers to a type of suicidal ideation in which the person has thoughts of killing oneself with details of plan fully or partially worked out and patient has some intent to carry it out (i.e., some degree of intent is implicit in the concept of plan).

In the present context, the term “major depressive disorder” or “MDD” (also known as clinical depression) refers to a psychiatric disorder characterized by at least two weeks of pervasive low mood, low self-esteem, and loss of interest or pleasure in normally enjoyable activities. The diagnosis of major depressive disorder is based on the person's reported experiences, behavior reported by relatives or friends, and a mental status examination. The course of the disorder varies widely, from a single episode lasting months to a lifelong disorder with recurrent major depressive episodes. The most widely used criteria for diagnosing depressive conditions are found in the American Psychiatric Association's Diagnostic and Statistical Manual of Mental Disorders (DSM) and the World Health Organization's International Statistical Classification of Diseases and Related Health Problems (ICD). Both DSM and ICD mark out typical (main) depressive symptoms. Under mood disorders, ICD-11 classifies major depressive disorder as either single episode depressive disorder (where there is no history of depressive episodes, or of mania) or recurrent depressive disorder (where there is a history of prior episodes, with no history of mania). ICD-11 symptoms, present nearly every day for at least two weeks, are a depressed mood or anhedonia, accompanied by other symptoms such as “difficulty concentrating, feelings of worthlessness or excessive or inappropriate guilt, hopelessness, recurrent thoughts of death or suicide, changes in appetite or sleep, psychomotor agitation or retardation, and reduced energy or fatigue.” These symptoms must affect work, social, or domestic activities. The ICD-11 system allows further specifiers for the current depressive episode: the severity (mild, moderate, severe, unspecified); the presence of psychotic symptoms (with or without psychotic symptoms); and the degree of remission if relevant (currently in partial remission, currently in full remission). These two disorders are classified as “Depressive disorders,” in the category of “Mood disorders.” According to DSM-5, there are two main depressive symptoms: a depressed mood, and loss of interest/pleasure in activities (anhedonia). These symptoms, as well as five out of the nine more specific symptoms listed, must frequently occur for more than two weeks (to the extent in which it impairs functioning) for the diagnosis. Major depressive disorder is classified as a mood disorder in DSM-5. The diagnosis hinges on the presence of single or recurrent major depressive episodes. Further qualifiers are used to classify both the episode itself and the course of the disorder. The category Unspecified Depressive Disorder is diagnosed if the depressive episode's manifestation does not meet the criteria for a major depressive episode.

In the present context, the term “major depressive episode” refers to a period characterized by the symptoms of major depressive disorder. Those affected primarily have a depressed mood for at least two weeks or more, and a loss of interest or pleasure in everyday activities. Other symptoms include feelings of emptiness, hopelessness, anxiety, worthlessness, guilt, irritability, changes in appetite, problems concentrating, remembering details or making decisions, and thoughts of suicide. Insomnia or hypersomnia, aches, pains, or digestive problems that are resistant to treatment may also be present. The description has been formalized in psychiatric diagnostic criteria such as the DSM-5 and ICD-11.

In the present context, the term “treatment-refractory depression” refers to a form of depression that responds poorly to currently available treatments (e.g., as described at nimh.nih.gov/trials/practical/stard/index.shtml June 2011) and which may have different underlying etiopathological mechanisms compared with other forms of depression. Combinations of antidepressants have typically not been shown to be superior to monotherapy for refractory depression and often increase risk of side effects and are not recommended.

In the present context, the term “effective amount” or a “therapeutically effective amount” of therapeutic agent(s) referenced herein, refers to a nontoxic, but sufficient amount of the same, to provide the desired effect. It is an amount sufficient to alleviate, arrest, partly arrest, remove, or delay the clinical manifestations of a given psychiatric disorder, and its complications in a therapeutic intervention including the administration of said compound(s). An amount adequate to accomplish this is defined as “therapeutically effective amount”. In a combination therapy of the present invention, an “effective amount” of one component of the combination is the amount of that compound that is effective to provide the desired effect when used in combination with the other components of the combination. Effective amounts for each purpose will depend e.g. on the severity of the disorder, as well as the weight and general state of the subject. It will be understood that determining an appropriate dosage may be achieved using routine experimentation, by constructing a matrix of values and testing different points in the matrix, which is all within the ordinary skills of a trained physician.

In the present context, the term “pharmaceutical product” or “combination product” refers to any product containing (i) nefazodone, or a pharmaceutically acceptable salt thereof and (ii) risperidone. The pharmaceutical product can be in the form of unit dose, such as tablets, capsules, oral soluble films, etc. The unit dose would contain (i) nefazodone, or a pharmaceutically acceptable salt thereof, (ii) risperidone, and (iii) one or more excipients. The pharmaceutical product is desirably in a unitary dosage form suitable for administration orally, nasally, rectally, percutaneously, or by parenteral injection.

In the present context, the term “unit dose” or “unit dosage form” refers to physically discrete units of a pharmaceutical product described herein, suitable as unitary dosages, each unit containing a predetermined quantity of (i) nefazodone, or a pharmaceutically acceptable salt thereof and (ii) risperidone, calculated to produce the desired therapeutic effect, in association with the required pharmaceutical carrier(s). It is especially advantageous to formulate the aforementioned pharmaceutical product in unit dosage form for case of administration and uniformity of dosage. The unit dose includes solid or liquid pharmaceutical dosage forms of (i) nefazodone, or a pharmaceutically acceptable salt thereof and (ii) risperidone, such as capsules, tablets, scored tablets, oral soluble films, lozenges, pills, gel capsules, granulates, powders, syrups, aqueous or non-aqueous solutions, suspensions, and sterile injectable solutions.

In the present context, the term “pharmaceutically acceptable salts” includes acid addition salts or addition salts of free bases. Examples of acids which may be employed to form pharmaceutically acceptable acid addition salts include, but are not limited to, organic salts such as maleic, fumaric, benzoic, ascorbic, embonic, succinic, oxalic, bis methylene-salicylic, methanesulfonic, ethanedisulfonic, acetic, propionic, tartaric, salicylic, citric, glucomic, lactic, malic, mandelic, cinnamic, citraconic, aspartic, stearic, palmitic, itaconic, glycolic, p-amino-benzoic, glutamic, benzene sulfonic, theophylline acetic acids as well as the 8-halotheophyllines (for example 8-bromo-theophylline) salts; and inorganic salts such as hydrochloric, hydrobromic, sulfuric, sulfamic, phosphoric and nitric acid salts.

In the present context, the term “antipsychotic drug” (also known as “neuroleptic”) refers to a class of psychotropic medication primarily used to manage psychosis (including delusions, hallucinations, paranoia, disordered thought, and other positive symptoms as defined in DSM-5), principally in schizophrenia but also in a range of other psychotic disorders. They are also the mainstay together with mood stabilizers in the treatment of bipolar disorder. Antipsychotic drugs include both typical and atypical antipsychotic drugs. Antipsychotic drugs are most frequently used for several conditions, e.g., schizophrenia, schizoaffective disorder, bipolar disorder (acute mania, mixed, and depressive episodes), psychotic depression, and treatment resistant depression.

Antipsychotic drugs

| | |
|---|---|
| Generic Name | |
| (Active Ingredient) | Brand Name |

Typical antipsychotics

| | |
|---|---|
| Acepromazine | Atravet, Acezine |
| Acetophenazine | Tindal |
| Benperidol | Frenactyl |
| Bromperidol | Bromidol, Bromodol |
| Butaperazine | Repoise, Tyrylen |
| Carfenazine | |
| Chlorproethazine | |
| Chlorpromazine | Largactil, Thorazine |
| Chlorprothixene | Cloxan, Taractan, Truxal |
| Clopenthixol | Sordinol |
| Cyamemazine | Tercian |
| Dixyrazine | Esucos |
| Droperidol | Droleptan, Dridol, Inapsine, Xomolix, Innovar |
| | (+Fentanyl) |
| Fluanisone | |
| Flupentixol | Depixol, Fluanxol |
| Fluphenazine | Prolixin, Modecate |
| Fluspirilene | Redeptin, Imap |
| Haloperidol | Haldol |
| Levomepromazine | Nosinan, Nozinan, Levoprome |
| Lenperone | Elanone-V |
| Loxapine | Loxapac, Loxitane |
| Mesoridazine | Serentil |
| Metitepine | |
| Molindone | Moban |
| Moperone | Luvatren |
| Oxypertine | Equipertine, Forit, Integrin, Lanturil, Lotawin, |
| | Opertil |
| Oxyprothepine | Meclopin |
| Penfluridol | Semap, Micefal, Longoperidol |
| Perazine | Taxilan |
| Periciazine | Neuleptil, Neulactil |
| Perphenazine | Trilafon |
| Pimozide | Orap |
| Pipamperone | Dipiperon, Dipiperal, Piperonil, Piperonyl, Propitan |
| Piperacetazine | Quide |
| Pipotiazine | Piportil |
| Prochlorperazine | Compazine, Stemzine, Buccastem, Stemetil, |
| | Phenotil |
| Promazine | Sparine |
| Prothipendyl | |
| Spiperone | Spiroperidol, Spiropitan |
| Sulforidazine | Imagotan, Psychoson, Inofal |
| Thiopropazate | Artalan, Dartal, Dartalan, Dartan |
| Thioproperazine | Majeptil |
| Thioridazine | Mellaril, Melleril |
| Thiothixene | Navane |
| Timiperone | |
| Trifluoperazine | Stelazine |
| Trifluperidol | |
| Triflupromazine | Vesprin |
| Zuclopenthixol | Clopixol |

Atypical antipsychotics

| | |
|---|---|
| Amoxapine | Asendin, Asendis, Defanyl, Demolox |
| Amisulpride | Amazeo, Amipride, Amival, Solian, Soltus, Sulpitac, |
| | Sulprix |
| Aripiprazole | Abilify |
| Asenapine | Saphris |
| Blonanserin | Lonasen |
| Brexpiprazole | Rexulti |
| Cariprazine | Vraylar |
| Carpipramine | Prazinil, Defekton |
| Clocapramine | Clofekton, Padrasen |
| Clorotepine | Clotepin, Clopiben |
| Clotiapine | Entumine |
| Clozapine | Clozaril |
| Iloperidone | Fanapt |
| Levosulpiride | |
| Lumateperone | Caplyta |
| Lurasidone | Latuda |
| Melperone | Bunil, Buronil, Eunerpan |
| Mosapramine | Cremin |
| Nemonapride | Emilace |
| Olanzapine | Zyprexa, Ozace, Lanzek, Zypadhera |
| Paliperidone | Invega |
| Perospirone | Lullan |
| Quetiapine | Seroquel |
| Remoxipride | Roxiam |
| Reserpine | Raudixin, Serpalan, Serpasil |
| Risperidone | Risperdal, Zepidone |
| Sertindole | Serdolect |
| Sulpiride | Sulpirid, Eglonyl |
| Sultopride | Barnetil, Barnotil, Topral |
| Tiapride | Equilium, Tiapridal |
| Veralipride | Agreal, Agradil |
| Ziprasidone | Geodon, Zeldox |

In the present context, the term “antidepressant drug” refers to a medication used to treat depression (e.g., major depressive disorder (MDD), some anxiety disorders, some chronic pain conditions, and to help manage some addictions (as defined in DSM-5), and further includes selective serotonin reuptake inhibitors (SSRIs), serotonin-norepinephrine reuptake inhibitors (SNRIs), serotonin modulators and stimulators (SMSs), serotonin antagonist and reuptake inhibitors (SARIs), norepinephrine reuptake inhibitors (NRIs), norepinephrine-dopamine reuptake inhibitors (NDRIs), tricyclic antidepressants (TCAs), tetracyclic antidepressants (TeCAs), and monoamine oxidase inhibitors (MAOIs).

Antidepressant drugs

| | |
|---|---|
| Generic Name | |
| (Active ingredient) | Brand Name |

Selective serotonin reuptake inhibitors (SSRIs)

| | |
|---|---|
| Citalopram | (Celexa, Cipramil) |
| Escitalopram | (Lexapro, Cipralex) |
| Fluoxetine | (Prozac, Sarafem) |
| Fluvoxamine | (Luvox, Faverin) |
| Paroxetine | (Paxil, Seroxat) |
| Sertraline | (Zoloft, Lustral) |

Serotonin-norepinephrine reuptake inhibitors (SNRIs)

| | |
|---|---|
| Desvenlafaxine | (Pristiq) |
| Duloxetine | (Cymbalta) |
| Levomilnacipran | (Fetzima) |
| Milnacipran | (Ixel, Savella, Milnaneurax) |
| Venlafaxine | (Effexor, Trevilor) |

Serotonin modulators and stimulators (SMSs)

| | |
|---|---|
| Vilazodone | (Viibryd) |
| Vortioxetine | (Trintellix, Brintellix) |

Serotonin antagonist and reuptake inhibitors (SARIs)

| | |
|---|---|
| Trazodone | (Desyrel) |
| Nefazodone | (Dutonin, Nefadar, Serzone) - withdrawn/discontinued |
| Etoperidone | (Axiomin, Etonin) - withdrawn/discontinued |

Norepinephrine reuptake inhibitors (NRIs)

| | |
|---|---|
| Reboxetine | (Edronax) |
| Teniloxazine | (Lucelan, Metatone) - also a 5-HT_{2A} receptor antagonist |
| Viloxazine | (Vivalan) - also a 5-HT_{2B} receptor antagonist and 5-HT_{2C} |
| | receptor agonist |
| Atomoxetine | (Strattera) - Off-label only |

Norepinephrine-dopamine reuptake inhibitors (NDRIs)

| | |
|---|---|
| Bupropion | (Wellbutrin, Elontril) - also a non-competitive antagonist of |
| | nicotinic acetylcholine receptors |
| Amphetamines | (e.g., Adderall, Dexedrine, Vyvanse) - actually |
| | norepinephrine-dopamine releasing agents (NDRAs) - Off- |
| | label only |
| Methylphenidate | (Ritalin) - Off-label only |
| Modafinil | (Provigil) - actually a selective dopamine reuptake inhibitor |
| | plus other actions - Off-label only |
| Amineptine | (Survector, Maneon) |
| Nomifensine | (Merital, Alival) |

Tricyclic antidepressants (TCAs)

| | |
|---|---|
| Amitriptyline | (Elavil, Endep) |
| Amitriptylinexide | (Amioxid, Ambivalon, Equilibrin) |
| Clomipramine | (Anafranil) |
| Desipramine | (Norpramin, Pertofrane) |
| Dibenzepin | (Noveril, Victoril) |
| Dimetacrine | (Istonil) |
| Dosulepin | (Prothiaden) |
| Doxepin | (Adapin, Sinequan) |
| Imipramine | (Tofranil) |
| Lofepramine | (Lomont, Gamanil) |
| Melitracen | (Dixeran, Melixeran, Trausabun) |
| Nitroxazepine | (Sintamil) |
| Nortriptyline | (Pamelor, Aventyl) |
| Noxiptiline | (Agedal, Elronon, Nogedal) |
| Opipramol | (Insidon) |
| Pipofezine | (Azafen/Azaphen) |
| Protriptyline | (Vivactil) |
| Trimipramine | (Surmontil) |
| Amineptine | (Survector, Maneon) and tianeptine (Stablon, Coaxil) are |
| | technically TCAs but are atypical, and are grouped elsewhere. |
| Butriptyline | (Evadyne) - withdrawn/discontinued |
| Demexiptiline | (Deparon, Tinoran) - withdrawn/discontinued |
| Fluacizine | (Phtorazisin) - withdrawn/discontinued |
| Imipraminoxide | (Imiprex, Elepsin) - withdrawn/discontinued |
| Iprindole | (Prondol, Galatur, Tetran) - withdrawn/discontinued |
| Metapramine | (Timaxel) - withdrawn/discontinued |
| Propizepine | (Depressin, Vagran) - withdrawn/discontinued |
| Quinupramine | (Kinupril, Kevopril) - withdrawn/discontinued |
| Tiazesim | (Altinil) - actually not a TCA but a tricyclic-like antidepressant - |
| | withdrawn/discontinued |
| Tofenacin | (Elamol, Tofacine) - actually not a TCA but a tricyclic-like |
| | antidepressant - withdrawn/discontinued |

Tetracyclic antidepressants (TeCAs)

| | |
|---|---|
| Amoxapine | (Asendin) |
| Maprotiline | (Ludiomil) |
| Mianserin | (Tolvon) |
| Mirtazapine | (Remeron) |
| Setiptiline | (Tecipul) |

Monoamine oxidase inhibitors (MAOIs)
Irreversible
Non-selective

| | |
|---|---|
| Isocarboxazid | (Marplan) |
| Phenelzine | (Nardil) |
| Tranylcypromine | (Parnate) |
| Benmoxin | (Neuralex) - withdrawn/discontinued |
| Iproclozide | (Sursum) - withdrawn/discontinued |
| Iproniazid | (Marsilid) - withdrawn/discontinued |
| Mebanazine | (Actomol) - withdrawn/discontinued |
| Nialamide | (Niamid) - withdrawn/discontinued |
| Octamoxin | (Ximaol) - withdrawn/discontinued |
| Pheniprazine | (Catron) - withdrawn/discontinued |
| Phenoxypropazine | (Drazine) - withdrawn/discontinued |
| Pivhydrazine | (Tersavid) - withdrawn/discontinued |
| Safrazine | (Safra) - withdrawn/discontinued |

Selective for MAO-B

| | |
|---|---|
| Selegiline | (Eldepryl, Zelapar, Emsam) |

Reversible
Non-selective

| | |
|---|---|
| Caroxazone | (Surodil, Timostenil) - withdrawn/discontinued |

Selective for MAO-A

| | |
|---|---|
| Metralindole | (Inkazan) - sometimes described as reversible inhibitors of |
| | MAO-A (RIMAs) |
| Moclobemide | (Aurorix, Manerix) - sometimes described as reversible |
| | inhibitors of MAO-A (RIMAs) |
| Pirlindole | (Pirazidol) - sometimes described as reversible inhibitors of |
| | MAO-A (RIMAs) |
| Eprobemide | (Befol) - withdrawn/discontinued |
| Minaprine | (Brantur, Cantor) - withdrawn/discontinued |
| Toloxatone | (Humoryl) - withdrawn/discontinued |

Mixed
Non-selective

| | |
|---|---|
| Bifemelane | (Alnert, Celeport) - RIMA, irreversible inhibitor of MAO-B, |
| | and weak NRI |

In the present context, the terms “treating” and “treatment” as used herein refer to a reduction in severity and/or frequency of symptoms, elimination of symptoms and/or underlying cause, prevention of the occurrence of symptoms and/or their underlying cause, and/or improvement or remediation of damage. As such, the treatment includes the management and care of a patient for the purpose of alleviating, arresting, partly arresting, removing and/or delaying progress of the clinical manifestation of the psychiatric disorder. Thus, for example, “treating” a patient involves prevention of a particular disorder or adverse physiological event in a susceptible individual as well as treatment of a clinically symptomatic individual. The patient to be treated is preferably a mammal, in particular a human being.

In the present context, the terms “treatment of a psychiatric disorder” and “treating a psychiatric disorder” (and terms “treatment of a psychiatric disease” and “treating a psychiatric disease”) mean administering a pharmaceutical product described herein to a person who has a psychiatric disorder with the result that said psychiatric disorder is reduced or mitigated, thereby leading to a lower frequency of psychosis or even to a complete prevention of the psychosis.

In the present context, the terms “treatment of suicidal behavior” and “treating suicidal behavior” mean administering a pharmaceutical product described herein to person who has a suicidal behavior with the result that said suicidal behavior is reduced or mitigated, thereby leading to a lower frequency of suicide behaviors or even to a complete prevention of the person committing or attempting to commit suicide behaviors.

In the present context, the terms “treatment of suicidal ideation” and “treating suicidal ideation” mean administering a pharmaceutical product described herein to a person who has a suicidal thoughts with the result that said suicidal thoughts are reduced or mitigated, thereby leading to a lower frequency of suicidal thoughts or even to a complete prevention of the person having suicidal thoughts. The terms refer to a reduction, mitigation, or elimination in suicidal thoughts in a person or patient as measured by the MADRS 10 or HAM-D items of suicidal thoughts, possibly leading to a lower frequency of suicide attempts.

In the present context, the terms “treatment of major depressive disorder” and “treating major depressive disorder” mean administering a pharmaceutical product described herein to a person who is suffering from major depressive disorder with the result that said depression is reduced or mitigated, thereby leading to a lower frequency of depression or even to a complete prevention of the person suffering from depression.

In the present context, the terms “subject,” “human subject,” “patient,” and “person” refer to a human being (e.g., child, adolescent, or adult) that is afflicted with a psychiatric disorder, or is at risk thereof. The terms include a human being individually, or as a population.

In the present context, the term “prescriber” refers to a medical professional (e.g., doctor, physician, physician assistant, psychiatrist, psychologist, nurse practitioner, etc.) who has the legal authority to prescribe the medication described herein, to be administered to the human subject, for the treatment of the psychiatric disorder.

In the present context, the term “advertising” refers to notifying, informing, and/or apprising one or more individuals of information (e.g., the efficacy of a pharmaceutical product for treating or reducing an indication), such as by mass media, including, but not limited to, newspaper, magazine, and internet advertisements, television commercials, and billboard signs. The term “advertising” as used herein also includes, including a statement that the pharmaceutical product can treat or reduce the indication in the labeling for the pharmaceutical product.

In the present context, the term “marketing” refers to the act or process of selling a product, including, but not limited to, any offer for sale or sale of a product.

In the present context, the term “maintenance drugs” refers to medications prescribed to treat chronic, long-term conditions and are taken on a regular, recurring basis. Examples of maintenance drugs are those used to treat depression, hypertension, and diabetes.

In the present context, the term “chronic” refers to lasting for a long period of time, of a long duration, continuing or occurring again and again for a long time.

In the present context, the term “long-term” refers to extending over a relatively long time period, occurring over, or involving, a relatively long period of time.

In the present context, the term “regular” refers to occurring at relatively fixed intervals; periodic, with normal frequency; not varying; relatively constant.

In the present context, the term “continuous” refers to without break, cessation, or interruption; without intervening space or time; uninterrupted; unbroken; continual; unceasing; constant; continued; protracted.

In the present context, the term “recurring” refers to happening or occurring frequently, with repetition.

In the present context, the term “acute therapy” or “acute care” refers to a therapy in which the patient receives urgent, short-term treatment for a severe disorder. This is a loading dose. In medical terms, care for acute health conditions is the opposite from chronic care, or longer-term care. Acute therapy is generally delivered by a team of medical professionals (e.g., doctor, physician, physician assistant, psychiatrist, psychologist, nurse practitioner, etc.). Acute therapy may require a stay in a hospital emergency department, psychiatric hospital (also known as mental health hospital or behavioral health hospital), urgent care center, or other short-term stay facility, along with follow-up outpatient care in the community.

In the present context, the term “ongoing maintenance,” “maintenance therapy” or “chronic therapy” refers to a therapy in which the patient receives long-term, continuous, and regular treatment for a chronic disorder. This is a maintenance dose. In medical terms, ongoing maintenance for chronic health conditions is the opposite from acute therapy. While ongoing maintenance can occur in a hospital emergency department, psychiatric hospital, urgent care center, and/or other short-term stay facility, the ongoing maintenance will typically occur as outpatient care.

In the present context, the term “finished pharmaceutical container” includes devices suitable for the storage of (1) solid pharmaceutical dosage forms such as capsules, tablets, oral soluble films, lozenges, pills, gel capsules, granulates, powders and the like and (2) liquid pharmaceutical dosage forms such as syrups, aqueous or non-aqueous solutions, suspensions, and the like, including sterile injectable solutions. The term encompasses, but is not limited to containers, blister packs, pill boxes, sachets, cassettes, bottles, jars, safety packings, tablet dispensers, ampoules, foil wrappings, pill or medicine organizers, dispensers and managers, pill fobs and totes.

In the present context, the term “written matter” encompasses, but is not limited to, package inserts, labels, patient brochures, patient leaflets, user manuals, and videotapes.

In the present context, the term “printed indicia” refers to marking(s) that indicate the marketing company name, the manufacturing company name, the drug substance name, the drug product name, the strength, the dosage form, the route of administration, and/or the product serialization.

In the present context, the term “tablet” refers to a pharmaceutical oral dosage form (oral solid dosage, or OSD) or solid unit dosage form that includes a medicament or medicaments with suitable excipients. It is manufactured from a mixture of active substances and excipients, usually in powder form, pressed or compacted from a powder into a solid dose. Tablets are prepared either by molding or by compression. The excipients can include diluents, binders or granulating agents, glidants (flow aids) and lubricants to ensure efficient tableting; disintegrants to promote tablet break-up in the digestive tract; sweeteners or flavors to enhance taste; and pigments to make the tablets visually attractive or aid in visual identification of an unknown tablet. A polymer coating is often applied to make the tablet smoother and easier to swallow, to control the release rate of the active ingredient, to make it more resistant to the environment (extending its shelf life), or to enhance the tablet's appearance. Medicinal tablets were originally made in the shape of a disk of whatever color their components determined, but are now made in many shapes and colors to help distinguish different medicines. Tablets are often stamped with symbols, letters, and numbers, which enable them to be identified. Sizes of tablets to be swallowed range from a few millimeters to about a centimeter. The tablet can be scored or unscored.

In the present context, the term “score” refers to a debossed line that runs across the planar surface of a solid oral dosage form (e.g., tablet), to facilitate splitting by breaking or cutting into smaller portions. This characteristic can be useful because the score can be used to facilitate the splitting of the tablet into fractions when less than a full tablet is desired for a dose. The fractions can be, e.g., of substantially equal size, such that relatively equal amounts of each of the two active ingredients can be administered over the divided doses. For example, with a scored oral tablet containing 125 mg nefazodone and 0.25 mg risperidone, each of the two fractions of the tablet will contain about 62.5 mg nefazodone and about 0.125 mg risperidone. Likewise, with a scored oral tablet containing 225 mg nefazodone and 0.50 mg risperidone, each of the two fractions of the tablet will contain about 112.5 mg nefazodone and about 0.25 mg risperidone. Additionally, with a scored oral tablet containing 275 mg nefazodone and 0.75 mg risperidone, each of the two fractions of the tablet will contain about 137.5 mg nefazodone and about 0.375 mg risperidone.

In the present context, the term “excipient” refers to an inactive substance (i.e., other than the active pharmaceutical ingredient(s)) used in the formulation of pharmaceutical product to bring functionality to the formulation. Suitable pharmaceutical excipients are described in, e.g., Handbook of Pharmaceutical Excipients, 9th Edition, edited by Paul J Sheskey, Bruno C Hancock, Gary P Moss, David J Goldfarb (2020). The desired function of an excipient is to guarantee the required biopharmaceutical and physicochemical properties of the pharmaceutical product. Also, excipients for tablets are known as auxiliary substances. According to British Pharmacopocia (BP), “Excipient is any constituent of a medicinal product that is not an active substance. Adjuvants, stabilizers, antimicrobial preservatives, diluents, antioxidants are excipients.” The ideal excipients will have the following characteristics: (1) An excipient will be physiologically inert; (2) Physically and chemically stable by themselves and in combination with active ingredient(s) or other excipients in a formulation; (3) Commercially available in an acceptable chemical and physical grade; (4) Compatible with active ingredient(s); (5) Nontoxic and acceptable by FDA or regulatory agencies; (6) It should have accepted organoleptic properties such as colorless or white to off-white color, odorless; (7) Economical (acceptably low); (8) Free from any impurities and microbial hazards; (9) They may not be contraindicated among them; (10) Do not hamper the bioavailability of active ingredients. Within the context of tablets, the below excipients are described.

In the present context, the term “diluents,” “fillers,” or “bulking agents” refers to excipients for tablets to increase the weight or volume. Diluents are fillers designed to make up the required bulk of the tablet when the drug dosage itself is inadequate to produce this bulk. Diluents are also known as fillers or bulking agents. Diluents provide improved cohesion, improve flow, allow direct compression manufacturing, and adjust tablet thickness or weight. Exemplary diluents used for solid oral dosages (e.g., tablets) include, e.g., microcrystalline cellulose; powdered cellulose [5-40% for wet granulation and 10-30% for dry granulation]; anhydrous lactose; lactose monohydrate; spray-dried lactose; mannitol; starch; pregelatinized starch; maize starch; corn starch; sorbitol; sucrose; compressible sugar (20-60%); confectioner's sugar (10-50%); sugar spheres; dextrates; dextrin; dextrose; calcium phosphate, dibasic, anhydrous; calcium carbonate; maltose; maltodextrin; kaolin; calcium phosphate, dibasic, dihydrate; tribasic calcium phosphate; calcium sulfate; cellaburate; calcium lactate; cellulose acetate; silicified microcrystalline cellulose; cellulose acetate; corn syrup; pregelatinized starch and corn starch; corn syrup solids; erythritol (30.0-90.0%); ethylcellulose (1.0-3.0%); ethyl acrylate and methyl methacrylate copolymer dispersion; fructose; isomalt; alpha-lactalbumin; lactitol; magnesium carbonate (direct compression ≤45); magnesium oxide; methacrylic acid and ethyl acrylate copolymer; methacrylic acid and methyl methacrylate copolymer; polydextrose; simethicone; pregelatinized modified starch; starch, pea; hydroxypropyl pea starch; starch, pregelatinized hydroxypropyl pea; potato starch; starch, hydroxypropyl potato; pregelatinized hydroxypropyl potato starch; starch, tapioca; wheat starch; starch hydrolysate, hydrogenated; pullulan; talc (5.0-30.0%); amino methacrylate copolymer; trehalose; and xylitol.

In the present context, the term “binder” or “binding agent” refers to excipients for tablets to facilitate the agglomeration of a powder into granules. According to USP, tablet binders are substances that are incorporated into formulations to facilitate the agglomeration of powder into granules during mixing with a granulating fluid such as water, hydroalcoholic mixtures, or other solvents. Tablet binders or binding agents are the substances that are added either dry or in liquid form during wet granulation to form granules or to promote cohesive compacts for directly compressed tablets. Binders are agents used to impart cohesive qualities to the powdered material. They impart cohesiveness to the tablet formulation that ensures the tablet remaining intact after compression, as well as improving the free-flowing qualities by the formulation of granules of desired hardness and size. Exemplary binders used for solid oral dosages (e.g., tablets) include, e.g., polyvinylpyrrolidone (also known as povidone); copovidone (2.0-5.0% in direct compression and 2.0-5.0% in wet granulation); carbomer (0.75-3.0%); corn starch and pregelatinized starch; pregelatinized starch (5-10%); carboxymethylcellulose sodium, carmellose sodium (1.0-6.0%); hypromellose/hydroxypropyl methylcellulose (HPMC), methocel (2-5%); PEG (polyethylene glycol); hydroxyethyl cellulose; hydroxypropyl cellulose (2.0-6.0%); hydroxyethylmethyl cellulose; calcium carboxymethylcellulose/calcium cellulose glycolate/carmellosum calcium (5-15%); guar galactomannan/guar gum (up to 10.0%); ethylcellulose; chitosan hydrochloride; dextrin; low-substituted hydroxypropyl cellulose; hydroxypropyl starch; ceratonia (0.15-0.75%); inulin; magnesium aluminum silicate (2.0-10.0%); maltodextrin (2-40% for direct compression and 3-10% for wet granulation); methylcellulose (1.0-5.0%); dextrates; polyethylene oxide (5.0-85.0%); povidone (0.5-5.0%);

sodium alginate (1.0-3.0%); starch (3-20% w/w usually 5-10%); liquid glucose (5.0-10.0%); sucrose (2-20% dry granulation and 50-67% wet granulation); compressible sugar (5-20% as a dry binder in tablet formulations); zein (30% for wet granulation); gelatin (1-3% for wet mix); polymethacrylates (10-35% for dry mix and 15-35% as a solution and 4.5-10.5% w/w solids); sorbitol (2-10% for wet mix); glucose (2-25% for wet mix); sodium alginate (1-3% for wet mix); zein; and acacia (1.0-5.0%).

In the present context, the term “disintegrant” refers to excipients for tablets to assist dosage form's breakup or disintegration into small units/fragments. A disintegrant is a substance or a mixture of substances added to a tablet to facilitate its breakup or disintegration into small units/fragments and allow a drug substance to fast dissolution. According to USP, disintegrants are functional components that are added to formulations to promote rapid disintegration into smaller units and to allow a drug substance to dissolve more rapidly. When disintegrants come in contact with water or stomach or intestinal fluid, they absorb liquid and start to swell, dissolve, or form gels. This causes the tablet structure to rupture and disintegrate, making increased surfaces for improved dissolution of the drug substance. Exemplary disintegrants used for solid oral dosages (e.g., tablets) include, e.g., crospovidone (commercial name-kollidon cl) (2-5%); croscarmellose sodium (commercial name ac-di-sol, primellose) (10-25% in capsules and 0.5-5.0% in tablets. 2% w/w is used in direct compressed tablets and 3% w/w in wet-granulation processed tablets.); low-substituted hydroxypropyl cellulose; sodium starch glycolate (commercial name primogel, explotab) (2-8%, optimum concentration is about 4%, although 2% is sufficient in many cases); chitosan hydrochloride; corn starch and pregelatinized starch; calcium alginate & calcium sodium alginate (<10%); docusate sodium (˜ 0.5%); microcrystalline cellulose (5-15%); hydroxypropyl starch; magnesium aluminum silicate (2-10%); methylcellulose (2.0-10.0%); sodium alginate (2.5-10%); starch (3-25% w/w); pregelatinized starch (5-10%); calcium carboxymethylcellulose/calcium cellulose glycolate/carmellosum calcium (1-15%); and powdered cellulose (5-20%).

In the present context, the term “lubricant” refers to excipients for tablets to reduce the frictional forces between particle-particle as well as particles and metal-contact surfaces. Lubricants are non-toxic, pharmacologically inactive substances added to the formulation to prevent adhesion of the tablet material to the surface of the dies and punches, reducing interparticle friction, facilitating the ejection of the tablets from the die cavity, and improving the rate of flow of the tablet granulation. According to USP, lubricants are substances that typically are used to reduce the frictional forces between particles and between particles and metal-contact surfaces of manufacturing equipment such as tablet punches and dies used in the manufacture of solid dosage forms. Before compaction, liquid lubricants may be absorbed into the tablet granule matrix. Exemplary lubricants used for solid oral dosages (e.g., tablets) include, e.g., magnesium stearate; magnesium silicate; calcium stearate; sodium lauryl sulphate; sodium stearyl fumarate; magnesium lauryl sulphate; stearic acid; calcium stearate; glyceryl behenate; behenoyl polyoxylglycerides; glyceryl dibchenate; lauric acid; glyceryl monostearate; glyceryl tristearate; myristic acid; palmitic acid; poloxamer; polyethylene glycol; polyethylene glycol 3350; polysorbate 20; polyoxyl 10 oleyl ether; polyoxyl 15 hydroxystearate; polysorbate 40; polyoxyl 20 cetostearyl ether; polyoxyl 40 stearate; polysorbate 60; polysorbate 80; potassium benzoate; sodium benzoate; sorbitan monolaurate; sorbitan monooleate; sodium stearate; sorbitan monopalmitate; sorbitan monostearate; zinc stearate; sorbitan sesquioleate; sorbitan trioleate; and talc.

In the present context, the term “glidant” and “anticaking agent” refers to excipients used to promote the flow properties of tablet granules or power materials. Glidants are non-toxic, pharmacologically inactive substance used to promote the flow properties of tablet granulation or powder materials by decreasing interparticle friction and cohesion. These always are added in the dry state during the lubrication step before compression. According to USP, glidants and anticaking agents are used to promote powder flow and to reduce the caking or clumping that can occur when powders are stored in bulk. Additionally, glidants and anticaking agents reduce the incidence of bridging during the emptying of powder hoppers and powder processing. Exemplary glidants used for solid oral dosages (e.g., tablets) include, e.g., colloidal silicon dioxide (trade name: acrosil 200/cab-o-sil); talc; tribasic calcium phosphate; calcium silicate; cellulose, powdered; magnesium oxide; sodium stearate; magnesium silicate; silica, dental-type; magnesium trisilicate; and hydrophobic colloidal silica.

In the present context, the term “coloring agents” or “colorant” refers to excipients used to give a color or identification of the tablets as either pigment or coating materials. Coloring agents are inactive substance(s) added into dosage forms to produce a distinctive appearance that may serve to differentiate a product from others that have a similar physical appearance or in some instances, to protect photolabile components of the dosage form. Coloring agents are categorized into: (1) Dyes: water-soluble coloring substances, (2) Lakes: insoluble forms of a dye that result from its irreversible adsorption onto a hydrous metal oxide, (3) Inorganic pigments: substances such as titanium dioxide or iron oxides, and (4) Natural colorants: colored compounds not considered dyes, such as riboflavin. Exemplary coloring agents used for solid oral dosages (e.g., tablets) include, e.g., caramel; ferric oxide; titanium dioxide; ferrosoferric oxide; aluminum oxide; FD & C red #40/allura red AC; amaranth; FD & C blue #1/brilliant blue FCF; canthaxanthin; carmine; carmoisine (azorubine); curcumin (tumeric); FD & C red #3/erythrosine; fast green FCF; green S (lissamine green); D & C red #30/helendon pink; FD & C blue #2/indigo carmine; iron oxide black; iron oxide red; D & C red #7/lithol rubin BK; patent blue V; D & C red #28/phloxine B; iron oxide yellow; D & C red #27/phloxine O; ponceau 4R (cochineal red A); quinoline yellow WS; D & C yellow #10; riboflavin (lactoflavin); FD & C yellow #5/tartrazine; and FD & C yellow #6/sunset yellow FCF.

In the present context, the term “flavoring agent” refers to excipients used in some types of tablets (e.g., chewable tablets or dispersible tablets) or in coating suspension to impart a pleasant flavor. According to USP, a flavor is a single chemical entity or a blend of chemicals of synthetic or natural origin that can produce a taste or aroma (i.e., fragrance) response when orally consumed or smelled. Flavoring agents are consumed orally and appreciated by both smell and taste while fragrances are only for external use and appreciated only by smell. Generally, flavors mask the unpleasant smell as well as taste and to make the product more palatable, thus increasing patient compliance. Exemplary flavoring agents used for solid oral dosages (e.g., tablets) include, e.g., vanillin; peppermint flavor powder; berry flavor powder; strawberry flavor powder; orange flavor powder; lemon flavor powder; orange essence; ethyl maltol; eucalyptus oil; isobutyl alcohol; sodium succinate; adipic acid; almond oil; anethole; benzaldehyde; denatonium benzoate; ethyl acetate; ethyl vanillin; ethylcellulose; fructose; fumaric acid; 1-glutamic acid, hydrochloride; lactitol; leucine; malic acid; maltol; menthol/racementhol; methionine; methyl salicylate; monosodium glutamate; peppermint oil; strawberry flavor; peppermint spirit; racemethionine; rose oil; rose water; sodium acetate; sodium lactate solution; tartaric acid; thymol; fumaric acid; inulin; isomalt; and neohesperidin dihydrochalcone.

In the present context, the term “sweetener” or “sweetening agent” refers to excipients used in some types of tablets (e.g., chewable tablets or dispersible tablets) or in coating suspension to impart a sweet flavor. Sweeteners are substances used to mask the unpleasant taste and sweeten oral dosage forms and also to mask unpleasant flavors. It binds to receptors on the tongue that are responsible for the sensation of sweetness. Sucrose is the standard for sweetness. Exemplary sweeteners used for solid oral dosages (e.g., tablets) include, e.g., sucralose; saccharin sodium; neotame; sucrose; acesulfame potassium; aspartame; aspartame acesulfame; corn syrup; corn syrup solids; dextrates; dextrose; dextrose excipient; erythritol; fructose; galactose; glucose; glycerin; inulin; invert sugar; isomalt; lactitol; maltitol; maltose; mannitol; saccharin; saccharin calcium; sorbitol; starch hydrolysate, hydrogenated; sugar, compressible; sugar, confectioner's; tagatose; trehalose; and xylitol.

In the present context, the term “surfactant” refers to excipients used for low solubility tablets to improve wetting and deagregation of drug particles to get a rapid and improved dissolution. Surfactants are substances with well-defined polar and non-polar regions that allow them to aggregate in solution to form micelles and non-polar drugs can partition into these micelles and be solubilized. They may decrease the surface tension (or interfacial tension) between a liquid and a solid or between a gas and a liquid or two liquids. Exemplary surfactants used for solid oral dosages (c.g., tablets) include, c.g., bchenoyl polyoxylglycerides; polysorbate 20; polysorbate 40; docusate sodium; polysorbate 60; polysorbate 80; benzalkonium chloride; caprylocaproyl polyoxylglycerides; cetylpyridinium chloride; lauroyl polyoxylglycerides; linoleoyl polyoxylglycerides; octoxynol 9; oleoyl polyoxylglycerides; poloxamer; polyoxyl 10 oleyl ether; polyoxyl 15 hydroxystearate; nonoxynol 9; polyoxyl 20 cetostearyl ether; polyoxyl 40 stearate; pullulan; polyoxyl lauryl ether; polyoxyl stearyl ether; sodium lauryl sulfate; sorbitan monolaurate; sorbitan monooleate; polyoxyl stearate; sorbitan monopalmitate; sorbitan monostearate; stearoyl polyoxylglycerides; sorbitan sesquioleate; sorbitan trioleate; and tyloxapol.

In the present context, the term “coating material” refers to excipients used as a film former to facilitate case in swallowing. These are substance used to coat tablets or particles. Coating materials are excipients for tablets, but not for all tablets. A proper coating formulation includes the following materials: film formers (which may be enteric or non-enteric); solvents; plasticizers; colorants; opaquant-extenders; and miscellaneous coating solution components.

### Specific Ranges, Values, and Embodiments

The specific embodiments provided below are for illustration purposes only, and do not otherwise limit the scope of the disclosed subject matter, as defined by the claims.

Methods of medical treatment

In specific embodiments, a unit dose containing nefazodone hydrochloride, risperidone, and one or more pharmaceutically acceptable excipients is administered to a person.

In specific embodiments, a unit dose containing nefazodone hydrochloride, risperidone, and one or more pharmaceutically acceptable excipients is administered to a person suffering from a psychiatric disorder or mental disorder.

In specific embodiments, a unit dose containing nefazodone hydrochloride, risperidone, and one or more pharmaceutically acceptable excipients is administered to a person suffering from a psychiatric disorder that includes suicidal behavior, suicidal ideation, and/or major depressive disorder (MDD).

In specific embodiments, a unit dose containing nefazodone hydrochloride, risperidone, and one or more pharmaceutically acceptable excipients is administered to a person suffering from suicidal behavior.

In specific embodiments, a unit dose containing nefazodone hydrochloride, risperidone, and one or more pharmaceutically acceptable excipients is administered to a person suffering from at least one of the subtypes of suicidal behavior: (i) suicide attempt, (ii) interrupted attempt, (iii) aborted attempt, and (iv) preparatory actions toward imminent suicidal behaviors.

In specific embodiments, a unit dose containing nefazodone hydrochloride, risperidone, and one or more pharmaceutically acceptable excipients is administered to a person suffering from the subtype of suicidal behavior, suicide attempt.

In specific embodiments, a unit dose containing nefazodone hydrochloride, risperidone, and one or more pharmaceutically acceptable excipients is administered to a person suffering from the subtype of suicidal behavior, interrupted attempt.

In specific embodiments, a unit dose containing nefazodone hydrochloride, risperidone, and one or more pharmaceutically acceptable excipients is administered to a person suffering from the subtype of suicidal behavior, aborted attempt.

In specific embodiments, a unit dose containing nefazodone hydrochloride, risperidone, and one or more pharmaceutically acceptable excipients is administered to a person suffering from the subtype of suicidal behavior, preparatory actions toward imminent suicidal behaviors.

In specific embodiments, a unit dose containing nefazodone hydrochloride, risperidone, and one or more pharmaceutically acceptable excipients is administered to a person suffering from suicidal ideation.

In specific embodiments, a unit dose containing nefazodone hydrochloride, risperidone, and one or more pharmaceutically acceptable excipients is administered to a person suffering from chronic suicidal ideation.

In specific embodiments, a unit dose containing nefazodone hydrochloride, risperidone, and one or more pharmaceutically acceptable excipients is administered to a person suffering from at least one of the subtypes of chronic suicidal ideation: (i) passive, (ii) active: nonspecific (no method, intent, or plan), (iii) active: method, but no intent or plan, (iv) active: method and intent, but no plan, and (v) active: method, intent, and plan.

In specific embodiments, a unit dose containing nefazodone hydrochloride, risperidone, and one or more pharmaceutically acceptable excipients is administered to a person suffering from the subtype of chronic suicidal ideation, passive.

In specific embodiments, a unit dose containing nefazodone hydrochloride, risperidone, and one or more pharmaceutically acceptable excipients is administered to a person suffering from the subtype of chronic suicidal ideation, active: nonspecific (no method, intent, or plan).

In specific embodiments, a unit dose containing nefazodone hydrochloride, risperidone, and one or more pharmaceutically acceptable excipients is administered to a person suffering from the subtype of chronic suicidal ideation, active: method, but no intent or plan.

In specific embodiments, a unit dose containing nefazodone hydrochloride, risperidone, and one or more pharmaceutically acceptable excipients is administered to a person suffering from the subtype of chronic suicidal ideation, active: method and intent, but no plan.

In specific embodiments, a unit dose containing nefazodone hydrochloride, risperidone, and one or more pharmaceutically acceptable excipients is administered to a person suffering from the subtype of chronic suicidal ideation, active: method, intent, and plan.

In specific embodiments, a unit dose containing nefazodone hydrochloride, risperidone, and one or more pharmaceutically acceptable excipients is administered to a person suffering from major depressive disorder (MDD).

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons at risk of committing suicide, persons having recently attempted to commit suicide, and/or persons having a history of suicide attempts.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons at risk of committing suicide.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons having recently attempted to commit suicide.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons having a history of suicide attempts.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes persons that are afflicted with treatment-refractory depression.

In specific embodiments, the person having suicidal thoughts and/or suicidal behavior suffers from depression.

In specific embodiments, the person having suicidal thoughts and/or suicidal behavior suffers from depression, including major or severe depression, depression with anxiety symptoms in the form of either anxiety disorders as defined in DSM-5 (Diagnostic and Statistical manual of Mental Disorders, Fifth Edition) or associated anxiety symptoms and in connection with other disorders involving depression.

In specific embodiments, the person having suicidal thoughts and/or suicidal behavior suffers from major or severe depression.

In specific embodiments, the person having suicidal thoughts and/or suicidal behavior suffers from major depressive disorder (MDD).

In specific embodiments, the person having suicidal thoughts and/or suicidal behavior suffers from a psychotic illness.

In specific embodiments, the person having suicidal thoughts and/or suicidal behavior suffers from a psychotic illness, such as schizophrenia or schizoaffective psychosis.

In specific embodiments, the person having suicidal thoughts and/or suicidal behavior suffers from depression and has previously undergone treatment with an antidepressant and/or an antipsychotic.

In specific embodiments, a unit dose containing nefazodone hydrochloride, risperidone, and one or more pharmaceutically acceptable excipients is administered to a person for the treatment, mitigation, and/or reduction of suicidal thoughts and/or suicidal behavior.

In specific embodiments, the person having suicidal thoughts and/or suicidal behavior fulfils the following inclusion criteria: (a) DSM-5 (i) major depression with symptoms present for a minimum of 4 weeks; (b) minimum total score of 22 on the 10-item MADRS (Montgomery SA, Asberg M., A new depression scale designed to be sensitive to change., Br. J. Psychiatry 1979, 134, 382-389) and a score of 2 or more on the HAM-D item (depressed mood) (Hamilton, M., Br. J. Soc. Clin. Psychol 1967, 6, 278-296). Persons who still fulfilled the MADRS and HAM-D criteria after 1-week, single-blind placebo lead-in were randomized to the double-blind treatment.

In specific embodiments, the person having suicidal thoughts and/or suicidal behavior fulfils the following inclusion criteria: (i) Subject meets Diagnostic and Statistical Manual of Mental Disorders (5th edition) (DSM-5) diagnostic criteria for a MDE, based upon clinical assessment and confirmed by the Mini International Psychiatric Interview (MINI); (ii) Subjects has chronic suicidal ideation, confirmed by a score of ≥3 (at least moderate) in the CGI-SS-r for at least 6 weeks (a suicidal ideation for most of the day, for more days than not) prior to screening; (iii) subject has a MADRS total score of ≥24 at Screening and Baseline; (iv) Subject is receiving a therapeutic dose of an antidepressant treatment (ADT) with or without augmentation for at least 8 weeks prior to screening; and (v) Subject can, in the opinion of the investigator, be safely managed as an outpatient for the entire treatment.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that meets the Diagnostic and Statistical Manual of Mental Disorders (5th edition) (DSM-5) diagnostic criteria for a MDE, based upon clinical assessment and confirmed by the Mini International Psychiatric Interview (MINI).

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that has chronic suicidal ideation, confirmed by a score of ≥3 (at least moderate) in the CGI-SS-r for at least 6 weeks (a suicidal ideation for most of the day, for more days than not) prior to screening.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that has a MADRS total score of ≥24 at Screening and Baseline.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that is receiving a therapeutic dose of an antidepressant treatment (ADT) with or without augmentation for at least 8 weeks prior to screening.

In specific embodiments, a unit dose containing nefazodone hydrochloride, risperidone, and one or more pharmaceutically acceptable excipients is administered to a person suffering from a psychiatric disorder, wherein the person suffering from a psychiatric disorder includes a person that can, in the opinion of the investigator, be safely managed as an outpatient for the entire treatment.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥1 (questionably suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥2 (mildly suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥3 (moderately suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥4 (markedly suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of ≥5 (severely suicidal).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 6 (among the most extremely suicidal patients).

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 1-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 2-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 3-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 4-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 5-6.

In specific embodiments, a unit dose containing nefazodone hydrochloride, risperidone, and one or more pharmaceutically acceptable excipients is administered to a person suffering from a psychiatric disorder having a Global Impression of Severity of Suicidality-revised (CGI-SS-r) score of 6.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤5.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤4.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤3.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person has a Global Impression of Severity of Suicidality-revised (CGI-SS-r) score of ≤2.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥1 unit in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥2 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥3 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of ≥4 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement of 5 units in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement to a score of 0 or 1 in the Global Impression of Severity of Suicidality-revised (CGI-SS-r) evaluation score.

In specific embodiments, a unit dose containing nefazodone hydrochloride, risperidone, and one or more pharmaceutically acceptable excipients is administered to a person suffering from a psychiatric disorder, such that during the period of treatment (or at the conclusion thereof), the person experiences an improvement to a score of 0 in the Global Impression of Severity of Suicidality-revised (CGI-SS-r) evaluation score.

In specific embodiments, the pharmaceutical product is a maintenance drug, prescribed to treat chronic, long-term psychiatric conditions and is taken on a regular, recurring, and ongoing basis. In those embodiments, the administration is long-term and continuous.

In specific embodiments, the period of treatment is more than 6 months.

In specific embodiments, the period of treatment is up to 6 months.

In specific embodiments, the period of treatment is up to 5 months.

In specific embodiments, the period of treatment is up to 4 months.

In specific embodiments, the period of treatment is up to 3 months.

In specific embodiments, the period of treatment is up to 2 months.

In specific embodiments, the period of treatment is up to 1 month.

In specific embodiments, the period of treatment up to 2 weeks.

In specific embodiments, the period of treatment is up to 1 week.

In specific embodiments, the treatment includes acute therapy followed by ongoing maintenance. In such embodiments, the period of treatment includes a period of acute therapy, followed by a period of ongoing maintenance. In such embodiments, the acute therapy and the maintenance therapy each independently include administering unit doses contains nefazodone (or a pharmaceutically acceptable salt thereof), risperidone, and one or more pharmaceutically acceptable excipients, in the strengths (i.e., amounts) described herein. In such embodiments, the unit dose(s) administered during the acute therapy can differ from the unit doses administered during the maintenance therapy. For example, relative to the unit dose(s) administered during the acute therapy, the unit doses administered during the maintenance therapy can contain nefazodone (or a pharmaceutically acceptable salt thereof) in a lower strength, can contain risperidone in a lower strength, or a combination thereof.

Additionally, in specific embodiments, the unit dose(s) administered during the acute therapy and the unit doses administered during the maintenance therapy can differ in their respective dosing regimens. For example, the unit dose(s) administered during the acute therapy can be twice daily (BID), while the unit doses administered during the maintenance therapy can be once daily (OD).

Additionally, in specific embodiments, the unit doses administered during the treatment can vary depending upon, e.g., a clinical evaluation carried out of the subject patient by a prescriber employing, e.g., CGI-SS-r. For example, the patient may receive an initial clinical evaluation (e.g., CGI-SS-r) and receive a rating of 6 (among the most extremely suicidal patients). From that initial rating, the patient may receive initial (acute therapy) unit doses that contain nefazodone (or a pharmaceutically acceptable salt thereof) and risperidone. The patient may receive a subsequent clinical evaluation (c.g., CGI-SS-r) and receive a rating of 3 (mildly suicidal). From that subsequent rating, the patient may receive subsequent (maintenance therapy) unit doses that contain nefazodone (or a pharmaceutically acceptable salt thereof) and risperidone. In specific embodiments, relative to the unit dose(s) administered during the acute therapy, the unit doses administered during the maintenance therapy can contain nefazodone (or a pharmaceutically acceptable salt thereof) in a lower strength, can contain risperidone in a lower strength, or a combination thereof. In additional specific embodiments, the unit dose(s) administered during the acute therapy can be twice daily (BID), while the unit doses administered during the maintenance therapy can be once daily (OD).

In specific embodiments, the period of acute therapy is up to 6 months.

In specific embodiments, the period of acute therapy is up to 5 months.

In specific embodiments, the period of acute therapy is up to 4 months.

In specific embodiments, the period of acute therapy is up to 3 months.

In specific embodiments, the period of acute therapy is up to 2 months.

In specific embodiments, the period of acute therapy is up to 1 month.

In specific embodiments, the period of acute therapy is up to 2 weeks.

In specific embodiments, the period of acute therapy is up to 1 week.

In specific embodiments, the period of ongoing maintenance is at least 1 month.

In specific embodiments, the period of ongoing maintenance is at least 2 months.

In specific embodiments, the period of ongoing maintenance is at least 3 months.

In specific embodiments, the period of ongoing maintenance is at least 4 months.

In specific embodiments, the period of ongoing maintenance is at least 5 months.

In specific embodiments, the period of ongoing maintenance is at least 6 months.

In specific embodiments, the period of ongoing maintenance is long-term and continuous.

In specific embodiments, the administration is a once-a-day (OD) administration.

In specific embodiments, the administration is a twice-a-day (BID) administration.

In specific embodiments, the method of treating a person suffering from a psychiatric disorder continues, provided the psychiatric disorder does not worsen.

In specific embodiments, the method of treating a person suffering from a psychiatric disorder continues, provided the severity of the psychiatric disorder remains about the same.

In specific embodiments, the method of treating a person suffering from a psychiatric disorder continues, provided the psychiatric disorder improves.

In specific embodiments, the method of treating a person suffering from a psychiatric disorder continues for a period of time effective to treat the psychiatric disorder, provided (i) the psychiatric disorder does not worsen, or (ii) the severity of the psychiatric disorder remains about the same, or (iii) the psychiatric disorder improves.

### Nefazodone Hydrochloride

In specific embodiments, the unit dose contains nefazodone (or a pharmaceutically acceptable salt thereof), risperidone, and one or more pharmaceutically acceptable excipients.

In specific embodiments, the nefazodone is present in the unit dose as the free base.

In specific embodiments, the nefazodone is present in the unit dose as a pharmaceutically acceptable salt thereof.

In specific embodiments, the nefazodone is present in the unit dose as the hydrochloride salt.

In specific embodiments, the nefazodone is present in the unit dose as the hydrochloride salt in 50-300 mg.

In specific embodiments, the nefazodone is present in the unit dose as the hydrochloride salt in 100-300 mg.

In specific embodiments, the nefazodone is present in the unit dose as the hydrochloride salt in 125-275 mg.

In specific embodiments, the nefazodone is present in the unit dose as the hydrochloride salt in 125±50 mg.

In specific embodiments, the nefazodone is present in the unit dose as the hydrochloride salt in 125±25 mg.

In specific embodiments, the nefazodone is present in the unit dose as the hydrochloride salt in 125±10 mg.

In specific embodiments, the nefazodone is present in the unit dose as the hydrochloride salt in 125±5 mg.

In specific embodiments, the nefazodone is present in the unit dose as the hydrochloride salt in 200±50 mg.

In specific embodiments, the nefazodone is present in the unit dose as the hydrochloride

salt in 200±40 mg.

In specific embodiments, the nefazodone is present in the unit dose as the hydrochloride salt in 200±25 mg.

In specific embodiments, the nefazodone is present in the unit dose as the hydrochloride salt in 200±10 mg.

In specific embodiments, the nefazodone is present in the unit dose as the hydrochloride salt in 225±75 mg.

In specific embodiments, the nefazodone is present in the unit dose as the hydrochloride salt in 225±50 mg.

In specific embodiments, the nefazodone is present in the unit dose as the hydrochloride salt in 225±25 mg.

In specific embodiments, the nefazodone is present in the unit dose as the hydrochloride salt in 225±10 mg.

In specific embodiments, the nefazodone is present in the unit dose as the hydrochloride salt in 225±5 mg.

In specific embodiments, the nefazodone is present in the unit dose as the hydrochloride salt in 275±75 mg.

In specific embodiments, the nefazodone is present in the unit dose as the hydrochloride salt in 275±50 mg.

In specific embodiments, the nefazodone is present in the unit dose as the hydrochloride salt in 275±25 mg.

In specific embodiments, the nefazodone is present in the unit dose as the hydrochloride salt in 275±10 mg.

In specific embodiments, the nefazodone is present in the unit dose as the hydrochloride salt in 275±5 mg.

### Risperidone

In specific embodiments, the unit dose contains nefazodone (or a pharmaceutically

acceptable salt thereof), risperidone, and one or more pharmaceutically acceptable excipients.

In specific embodiments, the risperidone is present in the unit dose in 0.20-1.0 mg.

In specific embodiments, the risperidone is present in the unit dose in 0.25-1.0 mg.

In specific embodiments, the risperidone is present in the unit dose in 0.25-0.75 mg.

In specific embodiments, the risperidone is present in the unit dose in 0.75±0.20 mg.

In specific embodiments, the risperidone is present in the unit dose in 0.75±0.15 mg.

In specific embodiments, the risperidone is present in the unit dose in 0.75±0.10 mg.

In specific embodiments, the risperidone is present in the unit dose in 0.75±0.075 mg.

In specific embodiments, the risperidone is present in the unit dose in 0.50±0.20 mg.

In specific embodiments, the risperidone is present in the unit dose in 0.50±0.10 mg.

In specific embodiments, the risperidone is present in the unit dose in 0.50±0.05 mg.

In specific embodiments, the risperidone is present in the unit dose in 0.25±0.1 mg.

In specific embodiments, the risperidone is present in the unit dose in 0.25±0.05 mg.

In specific embodiments, the risperidone is present in the unit dose in 0.25±0.025 mg.

### Combination

In specific embodiments, the nefazodone is present in the unit dose as the hydrochloride salt in 100-300 mg, and the risperidone is present in the unit dose in 0.25-1.0 mg.

In specific embodiments, the nefazodone is present in the unit dose as the hydrochloride salt in 125±15 mg, and the risperidone is present in the unit dose in 0.25±0.05 mg.

In specific embodiments, the nefazodone is present in the unit dose as the hydrochloride salt in 125±10 mg, and the risperidone is present in the unit dose in 0.25±0.03 mg.

In specific embodiments, the nefazodone is present in the unit dose as the hydrochloride salt in 125±5 mg, and the risperidone is present in the unit dose in 0.25±0.02 mg.

In specific embodiments, the nefazodone is present in the unit dose as the hydrochloride salt in 225±50 mg, and the risperidone is present in the unit dose in 0.50±0.05 mg.

In specific embodiments, the nefazodone is present in the unit dose as the hydrochloride salt in 225±25 mg, and the risperidone is present in the unit dose in 0.50±0.03 mg.

In specific embodiments, the nefazodone is present in the unit dose as the hydrochloride salt in 225±10 mg, and the risperidone is present in the unit dose in 0.50±0.02 mg.

In specific embodiments, the nefazodone is present in the unit dose as the hydrochloride salt in 275±75 mg, and the risperidone is present in the unit dose in 0.75±0.20 mg.

In specific embodiments, the nefazodone is present in the unit dose as the hydrochloride salt in 275±50 mg, and the risperidone is present in the unit dose in 0.75±0.10 mg.

In specific embodiments, the nefazodone is present in the unit dose as the hydrochloride salt in 275±25 mg, and the risperidone is present in the unit dose in 0.75±0.05 mg.

### Marketing & Advertising

In specific embodiments, the pharmaceutical product is marketed, advertised, and/or sold for the treatment, mitigation, and/or reduction of suicidal thoughts and/or suicidal behavior in a person in need thereof.

In specific embodiments, the pharmaceutical product is marketed, advertised, and/or sold for the treatment, mitigation, and/or reduction of depression in a person in need thereof.

In specific embodiments, the pharmaceutical product is marketed, advertised, and/or sold for the treatment, mitigation, and/or reduction of major depressive disorder (MDD) in a person in need thereof.

In specific embodiments, the pharmaceutical product is marketed, advertised, and/or sold to physicians treating people suffering from suicidal thoughts and/or suicidal behavior.

In specific embodiments, the pharmaceutical product is marketed, advertised, and/or sold to prescribers treating people suffering from suicidal thoughts and/or suicidal behavior.

In specific embodiments, the marketing includes the step of including a statement in the labeling for a pharmaceutical product that can treat, mitigate, and/or reduce suicidal thoughts and/or suicidal behavior in a person.

In specific embodiments, the marketing includes the step of including a statement in the labeling for a pharmaceutical product that can treat depression in a person in need thereof.

In specific embodiments, the marketing includes the step of including a statement in the labeling for a pharmaceutical product that can treat major depressive disorder (MDD) in a person in need thereof.

### Unit Dose and Excipients

In specific embodiments, the pharmaceutical product is a unit dose, formulated as an oral dosage form.

In specific embodiments, the pharmaceutical product is a unit dose, formulated as a solid oral dosage form.

In specific embodiments, the pharmaceutical product is a unit dose, formulated as a liquid oral dosage form.

In specific embodiments, the pharmaceutical product is formulated as a syrup, oral solution, or oral suspension.

In specific embodiments, the pharmaceutical product is formulated as an orally disintegrating tablet.

In specific embodiments, the pharmaceutical product is formulated as a lozenge.

In specific embodiments, the pharmaceutical product is formulated as an oral thin film.

In specific embodiments, the pharmaceutical product is formulated as a powder, effervescent powder, or effervescent tablet.

In specific embodiments, the pharmaceutical product is formulated as an oral tablet or an oral capsule.

In specific embodiments, the pharmaceutical product is formulated as an oral tablet.

In specific embodiments, the pharmaceutical product is formulated as an oral tablet that is scored.

In specific embodiments, the pharmaceutical product is formulated as an oral tablet that is not scored.

In specific embodiments, the pharmaceutical product is formulated as an oral capsule.

In specific embodiments, the pharmaceutical product is formulated as an oral tablet or as an oral capsule, and contains one or more diluents, one or more binders, one or more disintegrants, one or more lubricants, one or more glidants, one or more colorants, one or more flavoring agents, one or more sweeteners, one or more surfactants, one or more coating materials, or a combination thereof.

In specific embodiments, the pharmaceutical product is formulated as an oral tablet or as an oral capsule, and contains diluent, binder, disintegrant, lubricant, glidant, colorant, flavoring agent, sweetener, surfactant, coating material, or a combination thereof.

In specific embodiments, the pharmaceutical product is formulated as an oral tablet and contains one or more diluents, one or more disintegrants, and one or more lubricants.

### ENUMERATED EMBODIMENTS

Specific enumerated embodiments <1> to <50> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Embodiment 1

A unit dose that includes: (i) nefazodone, or a pharmaceutically acceptable salt thereof, (ii) risperidone, and (iii) one or more pharmaceutically acceptable excipients.

### Embodiment 2

The unit dose of embodiment <1>, wherein the nefazodone is present as the hydrochloride salt.

### Embodiment 3

The unit dose of embodiment <1>, wherein the nefazodone is present as the hydrochloride salt in 50-300 mg.

### Embodiment 4

The unit dose of embodiment <1>, wherein the nefazodone is present as the hydrochloride salt in 100-300 mg.

### Embodiment 5

The unit dose of embodiment <1>, wherein the nefazodone is present as the hydrochloride salt in 125-275 mg.

### Embodiment 6

The unit dose of any one of embodiments <1> to <5>, wherein the risperidone is present

in 0.20-1.0 mg.

### Embodiment 7

The unit dose of any one of embodiments <1> to <5>, wherein the risperidone is present in 0.25-1.0 mg.

### Embodiment 7

The unit dose of any one of embodiments <1> to <5>, wherein the risperidone is present in 0.25-0.75 mg.

### Embodiment 8

The unit dose of any one of embodiments <1> to <7>, wherein the unit dose is an oral dosage form.

### Embodiment 9

The unit dose of any one of embodiments <1> to <7>, wherein the unit dose is a solid oral dosage form.

### Embodiment 10

The unit dose of any one of embodiments <1> to <7>, wherein the unit dose is an oral tablet or an oral capsule.

### Embodiment 11

The unit dose of any one of embodiments <1> to <7>, wherein the unit dose is an oral tablet that is scored.

### Embodiment 12

The unit dose of any one of embodiments <1> to <7>, wherein the unit dose is an oral tablet that is not scored.

### Embodiment 13

The unit dose of any one of embodiments <1> to <7>, wherein the unit dose is a solid oral dosage form further including one or more diluents, one or more binders, one or more disintegrants, one or more lubricants, one or more glidants, one or more colorants, one or more flavoring agents, one or more sweeteners, one or more surfactants, one or more coating materials, or a combination thereof.

### Embodiment 14

A method of treating a psychiatric disorder in a human subject afflicted with the psychiatric disorder or is at risk thereof, the method including administering to the person the unit dose of any one of embodiments <1> to <13>.

### Embodiment 15

The method of embodiment <14>, wherein the psychiatric disorder includes suicidal behavior, suicidal ideation, and/or major depressive disorder (MDD).

### Embodiment 16

The method of embodiment <14>, wherein the psychiatric disorder includes suicidal behavior.

### Embodiment 17

The method of embodiment <14>, wherein the psychiatric disorder includes suicidal ideation.

### Embodiment 18

The method of embodiment <14>, wherein the psychiatric disorder includes major depressive disorder (MDD).

### Embodiment 19

The method of embodiment <14>, wherein the psychiatric disorder includes suicidal behavior that includes at least one of the subtypes of suicidal behavior: (i) suicide attempt, (ii) interrupted attempt, (iii) aborted attempt, and (iv) preparatory actions toward imminent suicidal behaviors.

### Embodiment 20

The method of embodiment <14>, wherein the psychiatric disorder includes chronic suicidal ideation that includes at least one of the subtypes of chronic suicidal ideation: (i) passive, (ii) active: nonspecific (no method, intent, or plan), (iii) active: method, but no intent or plan, (iv) active: method and intent, but no plan, and (v) active: method, intent, and plan.

### Embodiment 21

The method of any one of embodiments <14> to <20>, wherein the human subject is at risk of committing suicide, has recently attempted to commit suicide, and/or has a history of suicide attempts.

### Embodiment 22

The method of any one of embodiments <14> to <21>, wherein the psychiatric disorder includes treatment-refractory depression.

### Embodiment 23

The method of any one of embodiments <14> to <21>, wherein the psychiatric disorder includes depression.

### Embodiment 24

The method of any one of embodiments <14> to <21>, wherein the psychiatric disorder includes severe depression.

### Embodiment 25

The method of any one of embodiments <14> to <21>, wherein the psychiatric disorder includes depression with anxiety.

### Embodiment 26

The method of any one of embodiments <14> to <21>, wherein the human subject has previously undergone treatment with an antidepressant and/or an antipsychotic.

### Embodiment 27

The method of any one of embodiments <14> to <26>, wherein treating the psychiatric disorder effectively treats, mitigates, and/or reduces suicidal thoughts and/or suicidal behavior in the human subject.

### Embodiment 28

The method of any one of embodiments <14> to <27>, wherein the unit dose is administered to a human subject having a Global Impression of Severity of Suicidality-revised (CGI-SS-r) score of ≥1.

### Embodiment 29

The method of any one of embodiments <14> to <27>, wherein the unit dose is administered to a human subject having a Global Impression of Severity of Suicidality-revised (CGI-SS-r) score of ≥2.

### Embodiment 30

The method of any one of embodiments <14> to <27>, wherein the unit dose is administered to a human subject having a Global Impression of Severity of Suicidality-revised (CGI-SS-r) score of ≥3.

### Embodiment 31

The method of any one of embodiments <14> to <27>, wherein the unit dose is administered to a human subject having a Global Impression of Severity of Suicidality-revised (CGI-SS-r) score of ≥4.

### Embodiment 32

The method of any one of embodiments <14> to <27>, wherein the unit dose is administered to a human subject having a Global Impression of Severity of Suicidality-revised (CGI-SS-r) score of ≥5.

### Embodiment 33

The method of any one of embodiments <14> to <27>, wherein the unit dose is administered to a human subject having a Global Impression of Severity of Suicidality-revised (CGI-SS-r) score of 6.

### Embodiment 34

The method of any one of embodiments <14> to <33>, wherein after the administration the person experiences an improvement of >1 unit in the Global Impression of Severity of Suicidality-revised (CGI-SS-r) evaluation score.

### Embodiment 35

The method of any one of embodiments <14> to <33>, wherein after the administration the person experiences an improvement of >2 unit in the Global Impression of Severity of Suicidality-revised (CGI-SS-r) evaluation score.

### Embodiment 36

The method of any one of embodiments <14> to <33>, wherein after the administration the person experiences an improvement of >3 unit in the Global Impression of Severity of Suicidality-revised (CGI-SS-r) evaluation score.

### Embodiment 37

The method of any one of embodiments <14> to <33>, wherein after the administration the person experiences an improvement of ≥4 unit in the Global Impression of Severity of Suicidality-revised (CGI-SS-r) evaluation score.

### Embodiment 38

The method of any one of embodiments <14> to <33>, wherein after the administration the person experiences an improvement to a score of 0 or 1 in the Global Impression of Severity of Suicidality-revised (CGI-SS-r) evaluation score.

### Embodiment 39

The method of any one of embodiments <14> to <38>, wherein the administration is carried out daily, at least once a day (OD).

### Embodiment 40

The method of any one of embodiments <14> to <38>, wherein the administration is carried out daily, twice a day (BID).

### Embodiment 41

The method of any one of embodiments <14> to <40>, wherein the administration is carried out daily for at least one week.

### Embodiment 42

The method of any one of embodiments <14> to <40>, wherein the administration is carried out daily for at least 1 month.

### Embodiment 43

The method of any one of embodiments <14> to <42>, wherein the administration is carried out daily and the administration includes a daily loading dose and a daily maintenance dose.

### Embodiment 44

The method of any one of embodiments <14> to <42>, wherein the administration is carried out daily and the administration includes a daily loading dose followed by a daily maintenance dose wherein,
the loading dose includes the nefazodone (or a pharmaceutically acceptable salt thereof) in a higher strength relative to the maintenance dose, and
the loading dose includes the risperidone in a higher strength relative to the maintenance dose.

### Embodiment 45

The method of any one of embodiments <14> to <42>, wherein the administration is carried out daily and the administration includes a daily loading dose followed by a daily maintenance dose wherein,
the loading dose is administered twice a day (BID), and
the maintenance dose is administered once a day (OD).

### Embodiment 46

The method of any one of embodiments <14>to <45>, wherein the administration is carried out daily and continues provided (i) the psychiatric disorder does not worsen, (ii) the severity of the psychiatric disorder remains about the same, or (iii) the psychiatric disorder improves.

### Embodiment 47

The method of any one of embodiments <14>to <45>, wherein the administration is carried out daily and continues provided the psychiatric disorder does not worsen.

### Embodiment 48

The method of any one of embodiments <14>to <45>, wherein the administration is carried out daily and continues provided the severity of the psychiatric disorder remains about the same.

### Embodiment 49

The method of any one of embodiments <14>to <45>, wherein the administration is carried out daily and continues provided the psychiatric disorder improves.

### Embodiment 50

The method of any one of embodiments <43>to <45>, wherein
the loading dose is administered in a hospital emergency department, psychiatric hospital, urgent care center, or short-term stay facility, and
the maintenance dose is administered as a maintenance drug, on a regular, recurring, and long-term basis to the human subject in outpatient care.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

### EXAMPLES

The invention will be illustrated by the following non-limiting examples.

### Formulation Examples

### Example 1A: Components and Composition

### 200 mg Nefazodone Hydrochloride; 0.5 mg Risperidone; Oral Tablet

| % w/w | Ingredient | Function | mg/tablet |
|---|---|---|---|
| 37.00 | Nefazodone | Active | 200.00 |
| 0.09 | Risperidone | Active | 0.50 |
| 37.00 | Microcrystalline Cellulose | Filler/Diluent | 200.00 |
| 23.13 | Lactose Monohydrate | Filler/Diluent | 125.00 |
| | (Pharmatose DCL 15) | | |
| 0.93 | Croscarmellose Sodium | Disintegrant | 5.00 |
| | (Primellose) | | |
| 0.93 | Sodium Stearyl Fumarate | Lubricant | 5.00 |
| 0.93 | Colloidal Silicon Dioxide | Lubricant | 5.00 |

| | | |
|---|---|---|
| 100.00 | Total | 540.50 |

### Example 1B: Manufacturing Process and Controls

### 200 mg Nefazodone Hydrochloride; 0.5 mg Risperidone; Oral Tablet

1. Pass all the materials through Sieve 60 mesh equivalent.
2. Charge Risperidone and Microcrystalline Cellulose #1 into V-Blender and mix the material for 10 minutes.
3. Charge Microcrystalline Cellulose #2 into V-blender and mix the material for 10 minutes.
4. Charge Microcrystalline Cellulose #3 into V-blender and mix the material for 10 minutes.
5. Charge Microcrystalline Cellulose #4 into V-blender and mix the material for 10 minutes.
6. Charge Microcrystalline Cellulose #5 into V-blender and mix the material for 10 minutes.
7. Charge Microcrystalline Cellulose #6 into V-blender and mix the material for 10 minutes.
8. Charge Microcrystalline Cellulose #7 into V-blender and mix the material for 10 minutes.
9. Charge Microcrystalline Cellulose #8 into V-blender and mix the material for 10 minutes.
10. Charge Lactose Monohydrate, Croscarmellose Sodium and Sodium Stearyl Fumarate into V-blender and mix the material for 10 minutes.
11. Charge Microcrystalline Cellulose #9 and Nefazodone into V-blender and mix the material for 10 minutes.
12. Pass the blend from step 11 and colloidal silicon dioxide through Sieve 60 mesh equivalent.
13. Charge the material from Step 12 and colloidal silicon dioxide into V-blender and mix the material for 15 minutes.
14. Discharge the product from the Blender. Collect the Blend into labeled, tared containers double-lined with polyethylene bags.
15. Compact the blend from Step 14 using roller compaction process. Pass the compacted blend through 12 #sieve. Collect the Granules into labeled, tared containers double lined with polyethylene bags.
16. Compress the granules into suitable tablets with a target weight of 540.5 mg.

### Example 1C: Strengths of Oral Dosage Forms

The pharmaceutical product described herein can include the following strengths of risperidone and nefazodone, in the specified oral dosage forms.

| | | RISPERIDONE | | |
|---|---|---|---|---|
| | | 0.25 mg | 0.5 mg | 1.0 mg |
| NEFAZODONE | 50 | Solid oral or | Solid oral or | Solid oral or |
| | mg | Oral liquid | Oral liquid | Oral liquid |
| | 200 | Solid oral or | Solid oral or | Solid oral or |
| | mg | Oral liquid | Oral liquid | Oral liquid |
| | 300 | Solid oral or | Solid oral or | Solid oral or |
| | mg | Oral liquid | Oral liquid | Oral liquid |

### Example 1D: Strengths of Oral Dosage Forms

The pharmaceutical product described herein can include the following strengths of risperidone and nefazodone, in the specified oral dosage forms.

| | RISPERIDONE |
|---|---|
| | 0.25-1.0 mg |
| NEFAZODONE | Solid oral or |
| 50-300 mg | Oral liquid |

### Example 1E: Strengths of Oral Dosage Forms

The pharmaceutical product described herein can include the following strengths of risperidone and nefazodone, in the specified oral dosage forms.

| | | RISPERIDONE | | |
|---|---|---|---|---|
| | | 0.25 mg | 0.5 mg | 1.0 mg |
| NEFAZODONE | 50 | Solid oral or | Solid oral or | Solid oral or |
| | mg | Oral liquid | Oral liquid | Oral liquid |
| | 200 | Solid oral or | Solid oral or | Solid oral or |
| | mg | Oral liquid | Oral liquid | Oral liquid |
| | 300 | Solid oral or | Solid oral or | Solid oral or |
| | mg | Oral liquid | Oral liquid | Oral liquid |

### Example 1F: Dosing Regimens and Strengths

The pharmaceutical product described herein can be formulated in the following strengths of risperidone and nefazodone, to be administered with the specified dosing regimens.

| | | RISPERIDONE | | |
|---|---|---|---|---|
| | | 0.25 mg | 0.5 mg | 1.0 mg |
| NEFAZODONE | 50 | OD or BID | OD or BID | OD or BID |
| | mg | | | |
| | 200 | OD or BID | OD or BID | OD or BID |
| | mg | | | |
| | 300 | OD or BID | OD or BID | OD or BID |
| | mg | | | |

### Example 1G: Dosing Regimens and Strengths

The pharmaceutical product described herein can be formulated in the following strengths and dosage form of risperidone and nefazodone, to be administered with the specified dosing regimen.

| | RISPERIDONE |
|---|---|
| | 0.25-0.75 mg |
| NEFAZODONE | Scored Tablet; |
| 125-275 mg | BID |

### Example 1H: Dosing Regimens and Strengths

The pharmaceutical product described herein can be formulated in the following strengths and dosage form of risperidone and nefazodone, to be administered with the specified dosing regimen.

| | | RISPERIDONE | | |
|---|---|---|---|---|
| | | 0.25 mg | 0.5 mg | 0.75 mg |
| NEFAZODONE | 125 | Scored | Scored | Scored |
| | mg | Tablet; BID | Tablet; BID | Tablet; BID |
| | 225 | Scored | Scored | Scored |
| | mg | Tablet; BID | Tablet; BID | Tablet; BID |
| | 275 | Scored | Scored | Scored |
| | mg | Tablet; BID | Tablet; BID | Tablet; BID |

### Example 1I: Dosing Regimens and Strengths

The pharmaceutical product described herein can be formulated in the following strengths and dosage form of risperidone and nefazodone, to be administered with the specified dosing regimen.

### BID Dosing:

Product A: scored oral tablet, 125 mg nefazodone; 0.25 mg risperidone.
Product B: scored oral tablet, 225 mg nefazodone; 0.50 mg risperidone.
Product C: scored oral tablet, 275 mg nefazodone; 0.75 mg risperidone.

### EXPERIMENTAL EXAMPLES

Treatment of Chronic Suicidal Ideation and Behavior in Patients with Major Depressive Disorder (MDD).

### Health Care Provider:

Sandeep Gaonkar MD.
Conventions Psychiatry & Counseling
4300 Weaver Parkway,
Suite 100-A,
Warrenville, IL 60555

### Example 2: Case Study 1

### Case 1 Summary:

A 35-year-old married female and mother of three, presented to the outpatient clinic Jul. 16, 2019 to establish care with a new psychiatrist for management of her depression, mood swings, and persistent suicidal ideations. She reported that her current medications, bupropion XL 300 mg q daily, valproic acid 500 mg BID, quetiapine 200 mg daily, and venlafaxine XR 150 mg BID, modafinil 200 mg q AM and noon, buspirone 10 mg TID are not helping. She had been experiencing acute daily changes in mood consisting of unhappiness, irritability, and anger with lead to arguments with her husband and family. She had been suffering from persistent depression, self-harm, and suicidal impulses. She had three previous suicide attempts resulting in hospitalization, the most recent being six years prior.

Two years prior, she was hospitalized after the police were summoned to her home after she had continued agitation and had destroyed her husband's home office and broke a television. She was admitted with a diagnosis of major depression with psychotic features and began treatment with Lithium ER 450 mg BID and olanzapine ODT 5 mg BID. Upon discharge, her diagnosis was Axis 1: Bipolar Disorder and Axis 3: Migraines. She stepped down to PHP/IOP and discharged upon completion of the program to an outpatient psychiatrist whom she has since been seeing regularly. Under his care, she was tried on various medications including duloxetine, which was increased up to 90 mg daily, mixed amphetamine salts 30 mg BID, lamotrigine 100 mg daily, valproic acid, an unknown SSRI, and diazepam 10 mg TID. At some point she was evaluated by a neurologist who began treatment with topiramate. She discontinued the medication herself due to self-report of forgetfulness.

### Mental Status Exam:

During the Initial Psychiatric Evaluation, she was alert and oriented to person, place, time, and situation. Appearance: Looks stated age, obese, with casual dress, good hygiene and grooming.
Eye contact: Appropriate
Posture: Upright
Gait: Within normal limits
Motor activity: No tremors, extrapyramidal symptoms, dystonias, or Tardive dyskinesia
Affect: Decreased to constricted, tearful at times, congruent
Mood: Sad and anxious, ego-syntonic
Behavior: Cooperative
Speech: Normal rate, rhythm
Attention: Within normal limits
Memory: Intact for remote and recent
Abstract thinking: Within normal limits
Thought process: Coherent, logical, goal-directed
Thought content: No delusion, passive suicidal ideations without plan or intent
Perceptual disturbance: None
Judgment: Within normal limits
Insight: Fair

Past Psychiatric History: 3 previous hospitalizations. For more see above.

Past Social History: Unremarkable birth history with milestones met on time. She reports her childhood was “rough” due to molestation by her father. Her parents divorced when she was six years old. A year later her father passed away from stomach cancer. Her mother passed away in 2017 from lung cancer. She has a brother who she describes as “troubled.”

She is married to a Pakistani immigrant with whom she has a good relationship as he puts up with a lot from her.

No present or past history of substance use. No legal issues.

### Course

She was diagnosed with Major Depressive Disorder, Borderline Personality Disorder, Rule out Bipolar Disorder. Her bupropion XL, quetiapine, modafinil, and buspirone were discontinued. Valproic acid 500 mg BID was continued and venlafaxine XR was decreased to 75 mg BID. Nefazodone 100 mg BID and risperidone 0.5 mg q HS were initiated.

At her two-week follow-up appointment on Aug. 1, 2019, she was accompanied by her husband who provided additional emotional history on the patient where she became tearful as it was being described. She still felt anxious with sad mood but improved. Her suicidal thoughts were more controlled and had been less argumentative. As her symptoms continued, nefazodone was increased to 150 mg BID for one week, then to 200 mg BID thereafter.

On Aug. 23, 2019, the patient reported, “To my surprise, I have the first combination of medication that is helping.

After another month, she reported drowsiness suspected to be caused by the increase in nefazodone to 200 mg BID. It was then decreased to Serzone to 100 mg q AM and 200 mg q HS.

Upon follow-up on Oct. 15, 2019, she was doing very well despite some issues with focus and concentration and the possibility of stimulant initiation was discussed.

On Nov. 11, 2019, she returned very anxious with mood instability and more suicidal ideations. She revealed that she forgets to take nefazodone and had run out of the medication. She agreed to restart the treatment and return in a month for follow up.

Within a month on Jan. 21, 2020, she was doing well without any side effects on valproic acid 500 mg BID, venlafaxine XR 150 mg BID, and nefazodone 100 mg q Am, 200 mg q HS, and risperidone 0.5 mg q HS. She had gotten a new job, celebrated her 15th wedding anniversary, and was very happy. She continued expressing her surprise that she was no more suicidal which she felt would never go way.

She returned on Mar. 30, 2020 reporting not feeling well the last 2-3 weeks with increased sleep and overall poor mood. For increased depressive symptoms, nefazodone was increased to 200 mg BID to follow up in one month.

Modafinil 200 mg q AM was restarted a month later as methylphenidate was discontinued at her request due to continued issues with motivation, especially in the mornings. She also continues lamotrigine 25 mg BID, nefazodone 200 mg BID, risperidone 0.5 mg q HS, and venlafaxine XR 150 mg BID. And has been doing well.

She has been more compliant with medications as she has learned missing nefazodone and Risperdal makes her suicidal and impulsive.

### Example 3

### CASE 2 Summary:

A 59-year-old female presented to the outpatient psychiatric clinic accompanied by her husband and son after being referred by a friend. Her condition had not improved over the past several years despite ongoing psychiatric treatment including in-patient hospitalizations and multiple medication failures. She reported she had been, “feeling the stress”, experiencing racing and ruminating thoughts, difficulty with conversation, and panic. She expressed persistent suicidal ideation. She denied current or past self-injurious behavior.

She had three previous hospitalizations due to depression and suicidal ideation with anxiety and agitation. Her most recent admission was two years prior with a diagnosis of Major Depressive Disorder with psychotic features and a possible diagnosis of Bipolar Disorder. With multiple antidepressant failures in the past, she was started and stepped down to the day program on Lithium ER 450 mg BID and olanzapine ODT 5 mg BID. Upon completion of the program, she was referred to an outpatient psychiatrist whom she had since been seeing regularly. Under his care, she was tried on various medications including duloxetine, which was increased up to 90 mg daily, mixed amphetamine salts 30 mg BID, lamotrigine 100 mg daily, valproic acid, an unknown SSRI, and diazepam 10 mg TID. At some point, she was evaluated by a neurologist who began treatment with topiramate.

### Mental Status Exam:

Sensorium: Alert and oriented to person, place, time, situation
Appearance: Casually dressed, well kempt, appeared older than stated age, thin to normal body habitus
Affect: Constricted, tearful at times
Mood: Depressed
Speech: Normal rate, tone, and rhythm. Louder when irritable and argumentative
Thought Content: Passive suicidal ideation, no delusions.
Thought Process: Coherent, logical, mildly circumstantial.
Perception: No auditory or visual hallucinations
Memory: Intact
Abstract Thinking: Intact
Judgment: Fair to good
Insight: Good

### Social History

Growing up, she did not like herself often feeling uncomfortable. Although she had a fair relationship with her parents and two younger sisters, she dreaded that her parents would divorce. She felt she did not get complimented by her parents while growing up which continues to affect her self-esteem.

### Course

During her initial psychiatric evaluation, she was diagnosed with Bipolar Disorder, Unspecified; Rule out Amphetamine-induced psychosis due to her intermittent paranoia, impulsivity, and agitation. Her medications, mixed-amphetamine salts 30 mg BID and duloxetine were immediately discontinued and lamotrigine 150 mg q AM and diazepam 10 mg TID were continued from her previous regimen.

Upon her two-week follow up, she was anxious and still depressed expressing passive but bothersome suicidal thoughts. She was started on risperidone 0.5 mg q HS and vilazodone 10 mg q day. Two days later, she called and reported she was much better since beginning the new medications. A month later, clonazepam 1 mg TID was initiated, risperidone 0.5 mg q HS was continued, and vilazodone was increased to 20 mg. She was doing fairly well and had improved tremendously since her initial visit. She reported this was the best she had felt in a few years. Her lamotrigine was then increased to 100 mg BID for ongoing racing thoughts.

Within that month, she deteriorated with increased depression, pacing at times, increased stress due to her son going through a divorce, and a negative outlook towards life. She was expressing an inability to feel accomplished, feeling empty, and continued suicidal ideation. The diagnosis of Borderline Personality Disorder was suspected. Nefazodone was initiated at 100 mg q day for five days, increasing to 100 mg BID thereafter while vilazodone was discontinued.

Two weeks later, she was doing slightly better, and her suicidal ideations were reduced.

After a month, the racing thoughts persisted, and she was tearful due to emotional and social stressors of her son's marital issues. Her anxiety had slightly improved. Lamotrigine was discontinued and valproic acid was initiated at 250 mg BID.

Four and a half months after her Initial Psychiatric Evaluation, she reported this was the first time she had felt like herself and was tolerating the medications well without side effects. This was after she underwent hip surgery she otherwise would not have coped well with. She no longer reported depressed mood, suicidal ideation, or mood instability and had a stable relationship with family. Lisdexamphatamine 30 mg daily was initiated for difficulty with focus and concentration since the stabilization of her mood and eventually optimized to 50 mg with mood remaining stable.

She began feeling “up and down” and having suicidal ideation after eight months of stability. She unknowingly or misinterpreted recommendations having stopped taking nefazodone for several weeks. She restarted nefazodone and was slightly increased. Close follow up showed rapid disappearance of suicidal thoughts.

Upon follow up a month after the increase, she was on risperidone 0.5 mg q HS, nefazodone 200 mg q AM and 300 mg q HS, valproic acid 250 mg BID, clonazepam 1 mg TID, Vyvanse 50 mg q day. She is stable and doing well overall. She claims this is the first time in her life she has done well with no intrusive suicidal ideations.

### Example 4: Subject Population

Key inclusion and exclusion criteria for candidate subjects for the medical treatment described herein are provided below.

### Key Inclusion Criteria

Subject meets Diagnostic and Statistical Manual of Mental Disorders (5th edition) (DSM-5) diagnostic criteria for a MDE, based upon clinical assessment and confirmed by the Mini International Psychiatric Interview (MINI).
Subjects has chronic suicidal ideation, confirmed by a score of ≥3 (at least moderate) in the CGI-SS-r for at least 6 weeks (a suicidal ideation for most of the day, for more days than not) prior to screening.
Subject has a MADRS total score of ≥24 at Screening and Baseline
Subject is receiving a therapeutic dose of an antidepressant treatment (ADT) with or without augmentation for at least 8 weeks prior to screening.
Subject can, in the opinion of the investigator, be safely managed as an outpatient for the entire treatment.

### Key Exclusion Criteria

Subject has a current or prior DSM-5 diagnosis of a psychotic disorder, or an MDE with psychotic symptoms.
Subject meets the DSM-5 criteria for substance use disorder (except for nicotine or caffeine) within 6 months before screening.

Criteria used to identify candidate subjects to receive the medical treatment described herein are described below.

### Example 5: Clinical Global Impression of Severity of Suicidality-revised (CGI-SS-r)

Considering your total clinical experience with suicidal patients and all information now available to you, how suicidal is the patient at this time?
0. Normal, not at all suicidal
1. Questionably suicidal
2. Mildly suicidal
3. Moderately suicidal
4. Markedly suicidal
5. Severely suicidal
6. Among the most extremely suicidal patients

| SCALE | RATING | GUIDE TO RATING (CGI-SS-r) |
|---|---|---|
| 0 | Normal, not at all suicidal | Not suicidal |
| 1 | Questionably suicidal | Minimal ideations; little if any impulsivity for |
| | | suicide, few risk factors and many protective |
| | | factors; and no impact on function. |
| 2 | Mildly suicidal | Occasional ideations; little if any impulsivity for |
| | | suicide; few risk factors; adequate protective |
| | | factors and no or minimal impact on function. |
| 3 | Moderately suicidal | Intermittent ideations; with possible impulsivity |
| | | for suicide; may or may not have plan or recent |
| | | attempt*; several risk factors; protective factors |
| | | may outweigh risk factors and some impact on |
| | | function. |
| 4 | Markedly suicidal | Regular ideations with intent or potential for |
| | | impulsive actions for suicide; may or may not have |
| | | plan or recent attempt*; multiple risk factors |
| | | outweigh protective factors; and marked impact on |
| | | function. |
| 5 | Severely suicidal | Frequent ideations with intent; well worked out |
| | | suicide plan; may or may not have recent attempt*; |
| | | multiple risk factors outweigh protective factors; |
| | | and major impact on function. |
| 6 | Among the most extremely | Nearly constant suicidal ideations and intent; well |
| | suicidal patients | worked out plan and preparations underway or |
| | | recent attempt*; and severe impact on function. |

*Consider seriousness/lethality of any plan or suicide attempt in overall rating

### Example 6: FoST

Considering all of the information available to you, what is the patient's frequency of suicidal thinking at this time?
Never
Rarely
Sometimes
Often
Most of the time.
All of the time

### Example 7: Clinical Evaluation and Diagnosis of Depression

Patients were clinically evaluated for depression by medical personnel. A diagnosis of depression was arrived at by conducting a thorough clinical interview and assessment of the subject patient. This typically included a discussion of the individual's symptoms, medical and psychiatric history, and any current stressors or life events. They may also have used standardized questionnaires or rating scales to help evaluate the severity of symptoms. In some cases, diagnostic imaging or laboratory tests were used to rule out other possible causes of symptoms. Ultimately, the diagnosis of depression was based on the criteria outlined in the Diagnostic and Statistical Manual of Mental Disorders (DSM-5) or the International Classification of Diseases (ICD-11).

### Example 8: Questions Used to Identify a Suicidal Patient

Questions used to identify a suicidal patient include:
1. “Are you thinking about hurting yourself or attempting suicide?”
2. “Do you have a plan for how you would go about hurting yourself or committing suicide?”
3. “Do you have access to the means (e.g., pills, weapons) to hurt yourself or commit suicide?”
4. “Have you been feeling hopeless or helpless lately?”
5. “Do you feel like you would be better off dead or that the world would be a better place without you?”
6. “Have you been experiencing a significant loss or change in your life recently?”
7. “Are you experiencing overwhelming stress or emotional pain?”
8. “Are you experiencing severe depression or other mental health issues?”

### Example 9: Questionnaires Used to Diagnose Major Depression

Questionnaires used to diagnose major depression, include:
1. Beck Depression Inventory (BDI): A widely used self-report questionnaire that measures the severity of depression symptoms.
2. Hamilton Depression Rating Scale (HDRS): A clinician-administered questionnaire that rates the severity of depression symptoms on a scale of 0-52.
3. Patient Health Questionnaire (PHQ-9): A self-administered questionnaire that assesses the presence and severity of depression symptoms according to the criteria of the DSM.
4. Zung Self-Rating Depression Scale (SDS): A self-report questionnaire that assesses the presence and severity of depression symptoms on a scale of 20-80.
5. Center for Epidemiological Studies Depression Scale (CES-D): A self-report questionnaire that measures the presence and severity of depression symptoms in community samples.
6. Columbia Suicide Severity Rating Scale (C-SSRS): Rating scale designed to provide a prospective, standardized measure of suicidality. The scale allows clinicians and researchers alike to assess the severity and lethality of suicidal behaviors and ideations, and can be used to monitor treatment outcomes and establish suicide risk in a variety of research and clinical settings.

1. A method of reducing suicidal ideation and suicidal behavior in a person, the method comprising orally administering twice-a-day (BID) a unit dose comprising nefazodone hydrochloride and risperidone, wherein the person is suffering from a psychiatric disorder having a Global Impression of Severity of Suicidality-revised (CGI-SS-r) score of ≥2, wherein,
the method comprises a period of acute therapy, followed by a period of ongoing maintenance,
the strength of the unit doses administered during the acute therapy differs from the strength of the unit doses administered during the maintenance therapy,
the strength of the unit doses administered during the maintenance therapy can vary,
the acute therapy comprises a loading dose, and
the period of ongoing maintenance is long-term and continuous.

2. The method of claim 1, wherein the person is suffering from a psychiatric disorder having a Global Impression of Severity of Suicidality-revised (CGI-SS-r) score of ≥3.

3. The method of claim 1, wherein the person is suffering from a psychiatric disorder having a Global Impression of Severity of Suicidality-revised (CGI-SS-r) score of ≥4.

4. The method of claim 1, wherein the person is suffering from a psychiatric disorder having a Global Impression of Severity of Suicidality-revised (CGI-SS-r) score of ≥5.

5. The method of claim 1, wherein the person is suffering from a psychiatric disorder having a Global Impression of Severity of Suicidality-revised (CGI-SS-r) score of 6.

6. The method of claim 1, wherein the person is suffering from a psychiatric disorder for at least 6 weeks.

7. The method of claim 1, which effectively reduces the incidence of suicidal ideation and suicidal behavior in a person suffering from a major depressive episode.

8. The method of claim 1, which effectively reduces the severity of depressive symptoms in the person.

9. The method of claim 1, wherein the person is afflicted with treatment-refractory depression.

10. The method of claim 1, wherein the person was previously prescribed an antipsychotic drug.

11. The method of claim 1, wherein the person was previously prescribed an antidepressant drug.

12. The method of claim 1, wherein the person is afflicted with treatment-refractory depression.

13. The method of claim 1, wherein treatment comprises acute therapy, such that the unit dose is administered in a hospital emergency department, psychiatric hospital, urgent care center, or other short-term stay facility.

14. The method of claim 1, wherein the treatment comprises maintenance therapy, such that the unit dose is a maintenance drug, orally administered on a regular, recurring, and long-term basis to the person in outpatient care as an ongoing maintenance in reducing suicidal ideation and suicidal behavior.

15. The method of claim 1, wherein the unit dose is an oral tablet, an oral capsule, an oral soluble film, an oral solution, an oral suspension, an oral powder, or oral granules.

16. The method of claim 1, wherein the unit dose is an oral solid.

17. The method of claim 1, wherein during the period of treatment, or at the conclusion thereof, the person experiences an improvement of ≥2 units in the Global Impression of Severity of Suicidality-revised (CGI-SS-r) evaluation score.

18. The method of claim 1, wherein during the period of treatment, or at the conclusion thereof, the person experiences an improvement of ≥3 units in the Global Impression of Severity of Suicidality-revised (CGI-SS-r) evaluation score.

19. The method of claim 1, wherein during the period of treatment, or at the conclusion thereof, the person experiences an improvement of ≥4 units in the Global Impression of Severity of Suicidality-revised (CGI-SS-r) evaluation score.

20. The method of claim 1, wherein the unit dose comprises 50-300 mg nefazodone hydrochloride and 0.20-1.0 mg risperidone.

21. The method of claim 1, wherein the unit dose is a scored tablet comprising 50-300 mg nefazodone hydrochloride and 0.20-1.0 mg risperidone.

22. The method of claim 1, wherein the period of acute therapy is up to 2 weeks.

23. The method of claim 1, wherein the period of acute therapy is up to 1 month.

24. The method of claim 1, wherein the long-term and continuous period of ongoing maintenance is at least 6 months.
